(19) United States
(12) Patent Application Publication
(10) Pub. No.: US 2023/0220395 A1
(43) Pub. Date: Jul. 13, 2023

(54) METHODS AND COMPOSITIONS FOR TREATMENT OF AGE-RELATED MACULAR DEGENERATION

(71) Applicant: University of Massachusetts, Boston, MA (US)

(72) Inventors: Claudio Punzo, Brookline, MA (US); Shun-Yun Cheng, Westborough, MA (US)

(21) Appl. No.: 17/996,711

(22) PCT Filed: Apr. 20, 2021
(86) PCT No.: PCT/US2021/028156

§ 371 Date: Oct. 20, 2022

Related U.S. Application Data
(60) Provisional application No. 63/013,395, filed on Apr. 21, 2020.

(51) Int. Cl. C12N 15/113; A61K 31/506; A61K 31/216; A61K 31/42; A61K 31/202; A61P 27/02

(52) U.S. Cl. CPC C12N 15/1137; A61K 31/506; A61K 31/216; A61K 31/42; A61K 31/202; A61P 27/02

(57) ABSTRACT

Aspects of the disclosure relate to methods and compositions for treatment of certain ocular diseases and disorders, for example age-related macular degeneration (AMD). In some embodiments, the methods comprise administering a subject having AMD one or more therapeutic agents that modulate the mTORCl pathway (or a component thereof). The disclosure is based, in part, on methods for treating AMD in a subject by administering one or more kinase inhibitors, for example one or more serine/threonine kinase inhibitors. In some embodiments, at least one of the serine/threonine kinase inhibitors is a Ribosomal protein S6 kinase beta-1 (S6K1) inhibitor.

### RELATED APPLICATIONS

This application claims the benefit under 35 U.S.C. 119(e) of the filing date of U.S. Provisional Application Ser. No. 63/013,395, filed Apr. 21, 2020, entitled “METHODS AND COMPOSITIONS FOR TREATMENT OF AGE-RELATED MACULAR DEGENERATION”, the entire contents of which are incorporated herein by reference.

### BACKGROUND

Age-related macular degeneration is the leading cause of blindness in the elderly of the industrialized world. Disease generally initiates with the formation of “Drusen”, which are lipoprotein-rich deposits that form between the Bruch's membrane (BrM) and the retinal-pigmented epithelium (RPE) or between the RPE and the photoreceptor (PR) outer segments. Twenty percent of individual with drusen progress to the advanced forms of the disease, which is characterized by geographic atrophy (GA) of the RPE and the underlying PRs or by neovascular pathologies. The only treatment available to date is in regards to the neovascular pathology (also referred to as “wet AMD”), which uses anti-angiogenesis antibodies to inhibit the action of the “vascular endothelial growth factor” (VEGF). There is no treatment to prevent progression from the early disease stages to the advanced stages. Nor is there a treatment available for the advanced form of GA (often referred to as “dry” AMD).

### SUMMARY

Aspects of the disclosure relate to methods and compositions for treatment of certain ocular diseases and disorders, for example age-related macular degeneration (AMD). In some embodiments, the methods comprise administering a subject having AMD one or more therapeutic agents that modulate the mTORC1 pathway (or a component thereof).

The disclosure is based, in part, on methods for treating AMD in a subject by administering one or more kinase inhibitors, for example one or more serine/threonine kinase inhibitors. In some embodiments, at least one of the serine/threonine kinase inhibitors is a mammalian target of rapamycin complex 1 (mTORC1) inhibitor and/or a Ribosomal protein S6 kinase beta-1 (S6K1) inhibitor.

Accordingly, in some aspects, the disclosure relates to a method of inhibiting drusen formation in an ocular tissue, the method comprising administering to cells of the ocular tissue one or more inhibitors of mammalian target of rapamycin complex 1 (mTORC1).

In some aspects, the disclosure provides a method for treating age-related macular degeneration (AMD) in a subject, the method comprising administering to the subject one or more inhibitors of mTORC1.

In some aspects, the disclosure provides a method of inhibiting drusen formation in an ocular tissue, the method comprising administering to cells of the ocular tissue one or more inhibitors of Ribosomal protein S6 kinase beta-1 (S6K1).

In some aspects, the disclosure provides a method for treating age-related macular degeneration (AMD) in a subject, the method comprising administering to the subject one or more inhibitors of Ribosomal protein S6 kinase beta-1 (S6K1).

In some embodiments, an ocular tissue comprises Bruch's membrane tissue, retinal pigment epithelium (RPE) tissue, macula tissue, or a combination thereof. In some embodiments, an ocular tissue comprises photoreceptor cells, retinal pigment epithelial cells (RPEs), ganglion cells, or a combination thereof.

In some embodiments, administration comprises topical administration, intravitreal administration, subconjunctival injection, intrachoroid injection, systemic injection, or any combination thereof. In some embodiments, administration reduces drusen formation by about 2-fold, 3-fold, 5-fold, 10-fold, 50-fold, 100-fold, or more than 100-fold in the ocular tissue relative to ocular tissue that has not been administered the one or more S6K1 inhibitor. In some embodiments, methods further comprise a step of administering to the subject an effective amount of di-docosahexaenoic acid (DHA). In some embodiments, DHA is administered as dietary supplement.

In some embodiments, at least one S6K1 inhibitor is a small molecule, peptide, protein, antibody, or inhibitory nucleic acid.

In some embodiments, an inhibitory nucleic acid is a dsRNA, siRNA, shRNA, miRNA, ami-RNA, antisense oligonucleotide (ASO), or aptamer. In some embodiments, an inhibitory nucleic acid reduces or prevents expression of S6K1 protein. In some embodiments, an inhibitory nucleic acid binds to a nucleic acid encoding a S6K1 protein.

In some embodiments, a protein is a dominant negative S6K1 protein.

In some embodiments, a small molecule is PF-4708671, rosmarinic acid methyl ester (RAME), A77 1726, or a salt, solvate, or analogue thereof. In some embodiments, a small molecule is a selective inhibitor of S6K1. In some embodiments, a S6K1 inhibitor does not bind to or inhibit expression or activity of mammalian target of rapamycin 1 (mTORC1).

In some embodiments, ocular tissue is in vivo, optionally wherein the ocular tissue is present in a subject's eye.

### BRIEF DESCRIPTION OF DRAWINGS

FIG. 1 shows pathology distribution in mice with loss of TSC1 in rods and two normal copies of S6K1 (^{rod}TSC1^{-/-} S6K1^{+/+}), with loss of TSC1 in rods and loss of S6K1 (^{rod}TSC1^{-/-} S6K1^{-/-}), with loss of TSC1 in rods and loss of one copy of S6K1 (^{rod}TSC1^{-/-} S6K1^{-/+}), and with two normal copies of TSC1 and loss of S6K1 (^{rod}TSC1^{+/+} S6K1^{-/-}). Loss of S6K1 in the context of loss of TSC1 in rods prevents advanced pathologies.

FIG. 2 shows fundus images and retinal-pigmented epithelium flat mounts showing that mice with one copy of S6K1 in and loss of TSC1 (^{rod}TSC1^{-/-} S6K1^{-/+}) develop fundus pathologies (left) and GA as seen on flat mounts. In contrast, pathology was not observed in mice with loss of both TSC1 and S6K1 (^{rod}TSC1^{-/-} S6K1^{-/-}).

FIG. 3 shows deletion of S6K1 in the context of loss of TSC1 prevents accumulation of ApoE and complement factor H (CHF), which are both hallmarks of early-stage AMD.

FIGS. 4A-4H show RPE digestion of POSs is perturbed in ^{rod}Tsc1^{-/-} mice. FIG. 4A shows relative percentage of di-DHA PE(44;12) and PC(44:12) lipids from total retinal extracts of genotypes indicated at 2M. Bars show mean±S.E.M. (n=6-9 mice, 2 retinas per mouse; ****p<0.0001). FIG. 4B shows the same as in FIG. 4A with purified POSs pooled from 6 retinas per genotype. FIG. 4C shows POS clearance shown as percentage of remaining dots 3 hrs after peak shedding (ratio between 11 to 8 am) in 2M old mice that were fed a DHA or control diet between weaning to 2M. Shown are mean±S.E.M. (n=6 RPE flat mounts; **p<0.05; **p<0.01). FIG. 4D shows the same as in FIG. 4C with 6M old mice that were fed a DHA diet for only 2 weeks. Shown are mean±S.E.M. (n=6 RPE flat mounts; **p<0.01; ****p<0.0001). FIG. 4E shows RPE polynucleation (left) and hypertrophy (right) analyses of ^{rod}Tsc1^{-/-} mice that were fed a DHA or control diet between weaning to 6M. Bars are mean±S.E.M. (n=6 mice RPE flat mounts; *p<0.05; **p<0.01). FIG. 4F Representative fundus images of ^{rod}Tsc1^{-/-} mice on control (top row) or DHA (bottom row) diet from weaning onwards until time point indicated in panel (M: Months). FIG. 4G shows AMD related markers on retinal sections of ^{rod}Tsc1^{-/-} mice that were fed a DHA or control diet between weaning to 6M. Higher magnification of the region between arrowheads is shown on top of each panel. (nuclear stained with DAPI; cone sheets marked peanut agglutinin lectin (PNA); magenta: ZO1 marking RPE boundaries for ApoE and C3 panels and Phalloidin marking boundaries for ApoB and CFH panels). Scale bar=20 μm. (GCL: ganglion cell layer; RPE retinal-pigmented epithelium). Images are representatives of 3 independent experiments on 3 different animals per genotype. FIG. 4H shows the same experiment as in FIG. 4A after feeding mice a DHA diet from weaning onwards for 10. Bars show mean±S.E.M. (n=3 mice, 2 retinas per mouse; *p<0.05; **p<0.01; ****p<0.0001).

FIGS. 5A-5E show PKM2 and HK2 expression are increased PRs of AMD patients. FIG. 5A shows immunohistochemistry (IHC) showing increased expression of PKM2 and HK2 (purple) on retinal cross-sections. Increased expression is seen throughout the PR layer of AMD patients and particularly in cone inner segments (arrows) and cone pedicles (arrowheads). Dotted lines demark some of the cone inner segments in non-diseased individuals. Enzymatic reaction for immunohistochemistry was 6 minutes except for second panel of a non-diseased individual with the PKM2 antibody (30 min.; all non-diseased sections in panel A are from the same retina). Scale bars: 45 μm. FIG. 5B shows immunofluorescence for p-S6 (red; blue nuclear DAPI). Scale bars: 50 μm. (FIG. 5A and FIG. 5B) OS: outer segments; IS: inner segments; ONL: outer nuclear layer; INL: inner nuclear layer; IPL: inner plexiform layer; GCL: ganglion cell layer. FIG. 5C shows quantification of Western blots for p-S6 and PKM2 with retinas from 2M old mice (n=3) of genotypes indicated. On top are representative Western images for each protein plus the actin control Western. Results are shown as mean±S.E.M. (**P<0.01, ****P<0.0001). FIGS. 5D-5E show measurements of retinal lactate (FIG. 5D) and NADPH (FIG. 5E) levels at 2M of genotypes indicated (n=4 for lactate and n=8 for NADPH). Results are shown as mean±S.E.M. (*P<0.05, **P<0.01).

FIGS. 6A-6C show aged ^{rod}Tsc1^{-/-} mice develop GA and neovascular pathologies. FIG. 6A shows representative fundus images of littermate controls (top row) and ^{rod}Tsc1^{-/-} mice (bottom row) at ages indicated. FIG. 6B shows representative fundus fluorescein angiography (FFA: bottom row) images with corresponding fundus image (top row) at 18M of genotypes indicated. ^{rod}Tsc1^{+/+} mice show occasionally some microglia accumulation while all ^{rod}Tsc1+/− mice show microglia accumulation (arrowhead). ^{rod}Tsc1^{-/-} mice develop retinal folds (arrows), GA (as indicated), and neovascular pathologies (dotted line). FIG. 6C shows percentage distribution of phenotypes explained in (FIG. 6B) in ^{rod}Tsc1^{-/-} mice at indicated ages. Last two bars show control mice where only microglia accumulation is seen. Bars show percentage±M.O.E. Numbers in brackets: number of mice analyzed (M: months).

FIGS. 7A-7G show histological analyses of advanced AMD-like pathologies. FIG. 7A shows RPE and corresponding retinal flat mount of same eye, showing autofluorescent RPE cells and corresponding area with retinal folds marked with the letter (b), and an area of GA and corresponding PR atrophy marked with the letter (c). RPE whole mount is shown in left half and corresponding retina in right half of the panel. Scale bar=300 μm. FIG. 7B shows higher magnification of region in panel (A) marked with letter (b) showing autofluorescent RPE cells (arrowhead: left panel) that correspond to retinal folds (arrowhead: middle panel). Right panel shows higher magnification of a fold (different eye) with Iba-1 staining (red) marking microglia. Scale bars=50 μm. FIG. 7C shows higher magnification of area of GA marked in panel (A) with the letter (c) showing in gray scales loss of RPE cells (left panel) and retinal PRs (right panel; PR side up showing reduced nuclear DAPI density). Note that no folds are visible in the area of GA in panel A (letter c) meaning that folds are not required for the formation of GA. Scale bar=50 μm. Colors in (A-C) are as indicated by labels in panels. Annotation of colors for panel (A) is indicated in the first two images of panel (B) (blue: nuclear DAPI; green: autofluorescence (AF) or cone sheets marked by peanut agglutinin lectin (PNA); red: RPE boundaries marked by ZO1, cones marked by cone arrestin (CA) or microglia marked by Iba-1). FIG. 7D shows semithin section through intermediate stage of GA showing RPE atrophy with PRs still present. No fold is present in this area of RPE atrophy. FIG. 7E shows consecutive OCT images through area of GA identified by fundus (same eye as shown in FIGS. 6A-6B: 18M with GA), showing collapse of the outer nuclear layer (ONL: between dotted lines). FIG. 7F shows semithin sections of eye with GA shown in (FIG. 7E) showing multilayered RPE (white asterisk), RPE migration into the retinal proper (arrow), RPE atrophy (between arrowheads) and retinal angiogenesis (red arrows). As PRs die retinal folds flatten if they overlap with areas of GA. Reminiscence of retinal folds is indicated by dotted lines. Scale bars=20 μm. FIG. 7G shows RPE polynucleation and hypertrophy analyses. Top shows representative RPE image of cell boundaries marked by ZO1 (red signal) used for quantification analyses with output from the IMARIS software to identify cell shape, size and nuclei (blue signal: nuclear DAPI). Bottom shows quantification of RPE polynucleation (left) and distribution of RPE cell size (right). Bars show mean±S.E.M. (n=4 RPE flat mounts; *p<0.05;). Scale bar=10 μm.

FIGS. 8A-8G show AMD-like pathologies are dependent on the dose of activated mTORC1. FIG. 8A shows representative littermate fundus images of ^{rod}Tsc1^{+/+} ^{rod}Raptor^{+/+} (top panels), ^{rod}Tsc1^{-/-} ^{rod}Raptor^{+/-} (middle panels) and ^{rod}Tsc1^{-/-} ^{rod}Raptor^{-/-} (bottom panels) mice at ages indicated. Fundus of ^{rod}Tsc1^{-/-} are shown in FIG. 2 and FIG. 12. (M: Months). FIG. 8B shows percentage distribution of retinal pathologies scored with mice between 12-18M of age of genotypes indicated. ^{rod}Tsc1^{+/+} are shown in FIG. 6C. Graph shows percentage±M.O.E. Numbers in brackets: number of mice analyzed. FIG. 8C shows analyses of RPE polynucleation and RPE hypertrophy at 12M of genotypes indicated. Bars show mean±S.E.M. (n=4 mice). FIG. 8D shows quantification of retinal PKM2 (white) and p-S6 (grey) levels performed by Western blot in 2M old mice of genotypes indicated. Bars show mean±S.E.M. (n=3 mice). FIGS. 8E and 8F show retinal lactate (FIG. 8E) and NADPH (FIG. 8F) levels at 2M of age of genotypes indicated. Bars show mean±S.E.M. (n=4 for lactate and n=7 for NADPH). FIG. 8G shows immunofluorescence for ApoB, ApoE, C3 and CFH (green signal) on retinal sections of 12M old mice of genotypes indicated. Higher magnification of the region between arrowheads is shown on top of each panel. (Blue: nuclear DAPI; red: peanut agglutinin lectin to detect cone segment; magenta: ZO1 to visualize RPE in ApoE and C3 panels or Phalloidin to visualize RPE in ApoB and CFH panels. Images are representative of 3 independent experiments with 3 different animals. Scale bars=20 μm.

FIGS. 9A-9K show RPE digestion of POSs is perturbed in ^{rod}Tsc1^{-/-} mice. FIG. 9A shows representative immuno-fluorescence images of RPE whole mounts from 2M old ^{rod}Tsc1^{-/-} mice at time of day indicated showing delayed POS clearance by RPE cells (lower row) when compared to control mice (top row). POS are shown in green stained for RHODOPSIN while RPE cell boundaries are shown in red stained for ZO1 expression. Scale bar=10 μm. FIG. 9B shows quantification of the number of Rho positive dots per RPE cell over the course of the day from 2M old mice of genotypes indicated, obtained from immunofluorescence images as shown in FIG. 9A. Bars show mean±S.E.M. (n=6-8 RPE flat mounts; **p<0.01; ****p<0.0001). FIG. 9B shows delay of POS clearance shown as percentage of remaining dots 3 hrs after peak shedding (ratio between 11 to 8 am) in 2M old mice of genotypes indicated. Bars show mean±S.E.M. (n=6-8 RPE flat mounts; ***p<0.001; ****p<0.0001). FIG. 9D shows relative percentage of di-DHA PE(44;12) and PC(44:12) lipids from total retinal extracts of genotypes indicated at 2M. Bars show mean±S.E.M. (n=6-9 mice, 2 retinas per mouse; ****p<0.0001). FIG. 9E shows the same as in FIG. 9D with purified POSs pooled from 6 retinas per genotype. FIG. 9F shows POS clearance shown as percentage of remaining dots 3 hrs after peak shedding (ratio between 11 to 8 am) in 2M old mice that were fed a DHA or control diet between weaning to 2M. Shown are mean±S.E.M. (n=6 RPE flat mounts; **p<0.05; **p<0.01). FIG. 9G shows same as in FIG. 9F with 6M old mice that were fed a DHA diet for only 2 weeks. Shown are mean±S.E.M. (n=6 RPE flat mounts; **p<0.01; ****p<0.0001). FIG. 9H shows RPE polynucleation (left) and hypertrophy (right) analyses of ^{rod}Tsc1−/− mice that were fed a DHA or control diet between weaning to 6M. Bars are mean±S.E.M. (n=6 mice RPE flat mounts; *p<0.05; **p<0.01). FIG. 9I shows representative fundus images of ^{rod}Tsc1^{-/-} mice on control (top row) or DHA (bottom row) diet from weaning onwards until time point indicated in panel (M: Months). FIG. 9J shows AMD related markers on retinal sections of ^{rod}Tsc1^{-/-} mice that were fed a DHA or control diet between weaning to 6M. Proteins of interest indicated on top are shown in green. Higher magnification of the region between arrowheads is shown on top of each panel. (Blue: nuclear DAPI; red: cone sheets marked peanut agglutinin lectin (PNA); magenta: ZO1 marking RPE boundaries for ApoE and C3 panels and Phalloidin marking boundaries for ApoB and CFH panels). Scale bar=20 μm. (GCL: ganglion cell layer; RPE retinal-pigmented epithelium). Images are representatives of 3 independent experiments on 3 different animals per genotype. FIG. 9K shows the same experiment as in FIG. 9D after feeding mice a DHA diet from weaning onwards for 10. Bars show mean±S.E.M. (n=3 mice, 2 retinas per mouse; *p<0.05; **p<0.01; ****p<0.0001).

FIGS. 10A-10F show cones contribute differently than rods to disease. FIG. 10A shows representative fundus images at 12M (top) and percentage distribution of pathologies (graphs below: microglia accumulation: top left; retinal folds: top right; GA: bottom left; angiogenesis: bottom right) seen over time of genotypes indicated (M: months). Graphs show percentage±M.O.E. (n=10-15 mice). FIG. 10B shows coneTsc1^{-/-} mouse at 12M showing PR atrophy on retinal flat mount with retinal microglia migrating to the site of injury (left panel) and choroidal neovascularization on corresponding RPE flat mount of the same region (right panel). Eye corresponds to fundus of coneTsc1^{-/-} shown in FIG. 10A. Scale bar=50 μm. Colors are as indicated by labels in panels (blue: nuclear DAPI; green: cone sheets marked by peanut agglutinin lectin (PNA) or vascular marked with lectin B4 (lectin B4); red: microglia marked by Iba1 or RPE boundaries marked by ZO1). FIG. 10C shows a semithin section image of coneTsc1^{-/-} mouse at 12M showing large drusen-like deposit (see inset). Below: EM image of deposit and to the right higher magnification of boxed area in EM image. The BrM is marked by a double arrow. Arrowheads mark RPE basal fold and arrows mark translucent lipid vesicles. FIG. 10D shows large drusen-like deposits marked with letter (D) in ^{rod&cone}Tsc1^{-/-} mouse at 12M showing accumulation of ApoE (red signal). Enlarged area between arrowheads of left image is shown to the right in bright field. Scale bars=20 μm. Colors are as indicated by labels in panel (blue: nuclear DAPI; green: cone sheets marked by peanut agglutinin lectin (PNA); red: deposits positive for ApoE,). FIG. 10E shows EM images of coneTsc1^{-/-} mouse at 12M showing basal mounds (asterisk: larger mound; arrowheads: micro mounds), lipoprotein vesicles in the BrM (arrows), dysmorphic mitochondria (M) and membranous discs (MD). To the right: enlarged area of basal mound marked with asterisk on the left showing lipoprotein vesicles in BrM (arrows). FIG. 10F shows large GA area in ^{rod&cone}Tsc1^{-/-} mouse at 12M showing TUNEL positive RPE cells. Left panel shows RPE whole mount, while right panel shows higher magnification of GA area surrounded by dysmorphic RPE cells and TUNEL positive nuclei (arrowheads). Inset shows higher magnification of TUNEL positive nuclei. Scale bar=300 μm left panel, and 15 μm in right panel. Colors are as indicated by labels in panels (blue: nuclear DAPI; green: autofluorescence (AF) in left panel and apoptosis marked by TUNEL in right panel; red: RPE boundaries marked by Phalloidin).

FIGS. 11A-11C both PKM2 and HK2 expression are increased in PRs of AMD patients. (FIGS. 11A-11B) Immunofluorescence for PKM2 (FIG. 11A) and HK2 (FIG. 11B) expressions (green signal) in PRs of non-diseased human donor eyes (top rows) and AMD donor eyes (bottom rows). First two columns are donor retinas shown in FIG. 5. First column shows images at same signal intensity between non-disease and diseased tissue. Images in column 2-4 show scaled signal where PKM2 levels have been increased by a factor 2 in non-diseased tissue to better visualize the signal in PRs, while HK2 levels were scaled by a factor of 1.5 in non-diseased tissue. In both cases the baseline signal has also been slightly increased when compared to the panels that show the same intensity (compare diseased tissue from column 1 with 2). Signal is generally stronger in cone pedicels, cone inner segments or throughout the outer nuclear layer in eyes from AMD patients when compared to non-diseased controls. Panel in (FIG. 11A) and (FIG. 11B) within the same column are corresponding sections from the same donor retina. (Blue: nuclear DAPI; red: peanut agglutinin lectin to visualize cone segments; green: PKM2 or HK2 as indicated; F: female; M: male; yrs: years; ages of individuals are indicated in panels in years). FIG. 11C shows immunofluorescence with the same PKM2 antibody at different ages in mouse showing a decrease of the PKM2 signal with age. Far right: Western blot and quantification for PKM2 with retinas from 3M and 36M old mice showing that total levels decline with age. (n=6 retinas) (*p<0.05).

FIG. 12 shows representative fundus images of the same eye over time. ^{rod}Tsc1^{-/-} mice were imaged at indicated ages to trace disease progression within the same animal over time. C16 and C26 mice developed GA (dotted lines) and neovascular pathologies. C180 and C194 developed retinal folds and had microglia migrating into the subretinal space but did not develop advanced pathologies. ^{rod}Tsc1^{+/+} mice, C24 and C28, show normal fundus over time. Fundus fluorescein angiography images (right column) shown are of fundus image of the oldest age indicated.

FIGS. 13A-13D show retinal folds are often filled with microglia. FIGS. 13A-13D show ^{rod}Tsc1^{-/-} mice at 4M of age. FIG. 13A shows fundus image showing bright spots, which represent retinal folds and small white spots, which are microglia. FIG. 13B shows an image of OCT scan of eye shown in (FIG. 13A) along green arrow in (FIG. 13A). Three folds are visible (arrows) on OCT scan. FIG. 13C shows a zoomed in view on a retinal flat mount showing a fold filled with microglia (same panel as shown in FIG. 8B). FIG. 13D shows a cross-section of a fold showing microglia inside and migrating from the inner nuclear layer towards the PR layer. (C, D: Blue: nuclear DAPI; green: peanut agglutinin lectin marking cone sheets; red: Iba-1 marking microglia).

FIGS. 14A-14D show loss of Tsc1 in PRs does not lead to rapid PR degeneration. FIG. 14A shows analysis of ONL thickness at 18M. Each symbol is mean±S.E.M (n=6 retinas) (*p<0.05; **p<0.01; ***p<0.0001). FIGS. 14B-14D show analyses of PRs function over time showing average a-wave amplitude of the scotopic (FIG. 14B) and photopic (FIG. 14C) responses and c-wave ERG amplitudes (FIG. 14D). Bars show mean±S.E.M (n=5, 5, 6, 4, 9 for Cre− and 8, 4, 4, 6, 5 for Cre+ mice at 2M, 9M, 12M, 18M, and >20M respectively) (*p<0.05; **p<0.01).

FIGS. 15A-15C show p-S6 in RPE cells is independent of CRE activity and increases over time. FIG. 15A shows immunofluorescence for p-S6 (red signal) on RPE flat mounts of ^{rod}Tsc1−/− mice at 2M (top) and 15M (bottom). Few p-S6 positive RPE cells (arrowheads) are seen at 2M. Bottom right shows higher magnification of pS6 positive RPE cells. Scale bar=500 μm top panel on the left and 50 μm in bottom panel on the right. (Green: Phalloidin to highlight RPE cell boundaries). FIG. 15B shows retinal sections showing CRE-Recombinase staining (red signal) in photoreceptor layer (left) but not in p-S6 positive (green signal) RPE cells (arrowhead; see enlarged images to the right). Two different examples are shown. Due to the strong signal of p-S6 in the RPE, signal intensity for p-S6 was reduced on cross-sections showing also retina. Thus p-S6 in PRs appears weaker than normal. Nuclear DAPI (blue signal) and peanut agglutinin lectin (magenta signal) were removed from 50% of panel to better visualize red and green signals. Scale bars=20 μm. Red signal behind the RPE is due to the nature of the anti-CRE antibody, as it is a mouse monoclonal antibody highlighting therefore also endothelial cells. FIG. 15C shows quantification of p-S6 positive RPE cells at 2M and 15M of genotypes indicated. Bars show mean±S.E.M (n=4 mice).

FIGS. 16A-16D show ^{rod}Tsc1^{-/-} mice show early hallmarks of AMD. FIG. 16A shows immunofluorescence for ApoB, ApoE, C3 and CFH (green signal) on retinal sections of 15M old ^{rod}Tsc1^{-/-} mice. Higher magnification of the region between arrowheads is shown on top of each panel. (Blue: nuclear DAPI; red: cone sheets marked peanut agglutinin lectin (PNA); magenta: ZO-1 marking RPE boundaries for ApoE and C3 panels and Phalloidin marking boundaries for ApoB and CFH panels). Scale bar=20 μm. Images are representatives of 3 independent experiments on 3 different animal per genotype. FIG. 16B shows ultrastructural image showing undigested POS at the BrM, thickened BrM, neutral lipid droplets (L) in BrM and basal laminar deposit (BLamD). Enlarged image below shows area between arrowheads. FIG. 16C shows Semi-thin section showing basal mounds (asterisk) of different sizes (arrow: large basal mound). Higher magnification of region between arrowheads is shown below showing also micro-mounds (asterisks). Scale bar=20 μm. FIG. 16D shows RPE autofluorescence of genotypes indicated at 15M. ^{rod}Tsc1^{-/-} mice show more accumulation of lipofuscin (red signal). Autofluorescence was acquired with a Cy3 filter. (Blue: nuclear DAPI). Scale bar=20 μm.

FIGS. 17A-17D show similarities among coneTsc1^{-/-} mice, ^{rod}Tsc1^{-/-} mice & ^{cone&rod}Tsc1^{-/-} mice. FIG. 17A shows immunofluorescence for ApoB, ApoE, C3 and CFH (green signal) on retinal sections of 15M old ^{cone&rod}Tsc1^{+/+} control mice, coneTsc1^{-/-} mice, and ^{cone&rod}Tsc1^{-/-} mice. Higher magnification of the region between arrowheads is shown on top of each panel. (Blue: nuclear DAPI; red: peanut agglutinin lectin to detect cone segment; magenta: ZO1 to visualize RPE in ApoE and C3 panels or Phalloidin to visualize RPE in ApoB and CFH panels. Images are representative of 3 independent experiments with 3 different animals. Scale bars=20 μm. FIG. 17B shows a summary of ApoB, ApoE, C3 and CFH expression changes seen in the different genotypes at 15M and in the DHA feeding experiment. Expression levels are indicated by “+” signs. Levels are arbitrary based on visual analyses of antibody staining in 3 animals per genotype. FIG. 17C shows POS clearance in genotypes indicated at 2M. Shown is the percentage of remaining dots at 11 am. Loss of Tsc1 in cones also affects digestion of rod outer segments as assays were performed with an anti-rhodopsin antibody. Bars show mean±S.E.M. (n=6 RPE flat mounts). FIG. 17D shows relative percentage of di-DHA PE (44:12) and PC (44:12) lipids from total retinal extracts of genotypes indicated at 2M. Bars show mean±S.E.M. (n=8 for ^{cone&rod}Tsc1^{+/+}, n=6 for ^{rod}Tsc1^{-/-}, n=5 for coneTsc1^{-/-}, n=3 for ^{cone&rod}Tsc1^{-/-} with 2 retinas per sample from the same animal).

FIG. 18 shows a schematic of two-stage disease progression. In the aging eye lipoproteins accumulate within the BrM (left side of image) as part of the normal aging process. In some individuals the accumulation of lipoproteins starts to exceed the normal age-related buildup resulting in the formation of a lipid wall (stage 1) at the RPE-BrM interphase. This stage is driven by environmental risk factors such as smoking, diet, lack of exercise and genetic risk factors that affect metabolism. Once the lipid barrier becomes too thick, glucose transfer from the choroidal vasculature to PRs is reduced. This results in a metabolic switch in PRs which initiates the second stage of the disease. This leads to increased accumulation of lipoproteins, changes in the expression of complement components and a reduction of retinal di-DHA PE and PC lipids. The initiation of this disease stage adds new risk alleles such as those of the complement system and immune system. Eventually, in some individuals the pathologies progress to GA or choroidal neovascularization.

FIGS. 19A-19F show the loss of TSC2 in rods (^{rod}TSC2^{-/-}) resulted in same overall pathologies as seen with loss of TSC1 in rods. FIG. 19A is a western blot image for p-S6 (black bars) and PKM2 (white bars) showing overall increased levels in ^{rod}TSC2^{-/-} mice. FIG. 19B shows fundus pathologies seen in ^{rod}Tsc2^{-/-} mice over time. Arrows under 9M indicate retinal folds and arrows under 12M and 18M indicate GA or neovascular (angiogenesis) pathologies. FIG. 19C shows no pathologies were seen in control litter mates. FIG. 19D shows percentage distribution of pathologies in ^{rod}Tsc2^{-/-} mice over time (months, M) and littermate controls at 18M. Each bar shows percentage of mice±M.O.E. Number in parentheses are number of mice analyzed. FIG. 19E shows fundus (left) and RPE flat mount (right; ZO1: top right panel) images show different GA formation development in ^{rod}Tsc2^{-/-} mice at 12M. Slow intermediate GA (top), severe circular formation of GA (middle) and irregular patch of GA (bottom). Arrows: GA sites. FIG. 19F shows immunofluorescence for ApoB, ApoE, C3 and CFH on retinal sections of 12M old mice of genotypes indicated. Similar to loss of TSC1 in rods loss of TSC2 leads to accumulation of lipoproteins (ApoE, ApoB), complement factor H (CFH) and loss of complement factor C3. Higher magnification of the region between arrowheads is shown on top of each panel. (Scale bars: 50 μm).

FIGS. 20A-20D show loss of TSC2 in rods (^{rod}Tsc2^{-/-}) resulted in same overall pathologies as seen with loss of TSC1 in rods. FIG. 20A shows representative images of RPE flat mount at 8 am and 11 am show accumulation of shed POS in both ^{rod}Tsc2^{+/+} and ^{rod}Tsc2^{-/-} mice at 2M. (Rhodopsin and ZO-1; Scale bar=50 mm). At 11 am there are many more POSs still present in ^{rod}Tsc2^{-/-} mice. FIG. 20B shows quantification of remaining POSs/RPE cell at 8 and 11 am. FIG. 20C shows percentage of phospholipids of retinal lipid profiling in showing a reduction in double DHA containing PE and PC lipids in ^{rod}Tsc2^{-/-} mice. Similar data was seen with loss of TSC1 in rods. FIG. 20D shows ERG recordings indicating increased scotopic rod responses in ^{rod}Tsc2^{-/-} mice, similar to what was seen in ^{rod}Tsc1^{-/-} mice. Photopic ERG recordings are unchanged between ^{rod}Tsc2^{+/+} and ^{rod}Tsc2^{-/-} mice. Bars show average a wave amplitude (μV)±S.E.M. (n=8 & 14 mice).

FIGS. 21A-21G show Loss of TSC2 and HK2 in rods (^{rod}Tsc2^{-/-} ^{rod}HK2^{-/-}) still results in same overall pathologies as seen with loss of TSC2 in rods. FIG. 21A is a lactate assay with 2 months old mice showing that retinal lactate levels return to normal in ^{rod}Tsc2^{-/-} ^{rod}HK2^{-/-} mice or in mice where mTORC1 activity is blocked (loss of Raptor: ^{rod}Tsc2^{-/-} ^{rod}Raptor^{-/-}). Each bar shows relative fold change compare to each wild-type littermate controls±S.E.M. (N=4-6 mice). FIG. 21B shows percentage distribution of pathologies at 12 and 18 months of age in ^{rod}Tsc2^{-/-} ^{rod}HK2^{-/-} and littermate controls. Each bar shows percentage of mice±M.O.E. Number in parentheses are number of mice analyzed. FIG. 21C shows example of GA and neovascular pathology in ^{rod}Tsc2^{-/-} ^{rod}HK2^{-/-} mice. First panel shows fundus. Second panel shows fundus fluorescein angiography (FFA) to detect the neovascular pathology. Third panel shows optical coherence tomography (OCT) of area where blood was leaking, showing sub-RPE edema and new blood vessels migrating into the retina. Last panel shows higher magnification of the RPE flat mount from the same eye showing in red blood vessels that have developed marked with IB-4. FIG. 21D shows example of ApoE positive drusen like deposit in brightfield and fluorescence in ^{rod}Tsc2^{-/-} ^{rod}HK2^{-/-} mice. FIG. 21E shows immunofluorescence for ApoE, C3 and CFH on retinal sections of aged mice of genotypes indicated. Similar to FIGS. 19A-19F, ^{rod}Tsc2^{-/-} ^{rod}HK2^{-/-} mice still showed accumulation of ApoE, CFH and a reduction in C3. Higher magnification of the region between arrowheads is shown on top of each panel. (Scale bars: 50 μm). FIG. 21F shows photoreceptor outer segment (POS) digestion assay as shown in FIGS. 20A-20D. There was a 37% increase in undigested POS in ^{rod}Tsc2^{-/-} ^{rod}HK2^{-/-} at 11 am (3 hours after the peak of POS shedding). Interestingly, or ^{rod}Tsc2^{-/-} ^{rod}HK2^{-/-} shed fewer outer segments that the Cre⁻ littermate control mice (black bars). FIG. 21G shows scotopic and photopic electroretinogram in ^{rod}Tsc2^{-/-} ^{rod}HK2^{-/-} mice indicating that the increase seen in ^{rod}Tsc2^{-/-} mice is reversed in ^{rod}Tsc2^{-/-} ^{rod}HK2^{-/-} mice. Bars show average a wave amplitude (μV)±S.E.M. (n=9 & 11 mice).

FIGS. 22A-22B show loss of TSC1 and Rictor in rods (^{rod}Tsc1^{-/-rod} Rictor^{-/-}) still resulted in same overall pathologies as seen with loss of TSC1 in rods. FIG. 22A shows examples of fundus images in 18 months old mice. Genotypes are indicated in each fundus. FIG. 22B shows percentage distribution of pathologies at 18 months of age in ^{rod}Tsc1^{-/-rod} Rictor^{-/-} and heterozygous (^{rod}Tsc1^{-/-rod} Rictor^{-/+}) littermate control mice. Heterozygous as well as homozygous Rictor loss of function mice still develop the same pathologies at a similar frequency than ^{rod}Tsc1^{-/-} mice.

FIGS. 23A-23B show distribution of pathologies seen at 12 months of age (GA and CNV: angiogenesis) in ^{rod}Tsc1^{-/-} S6K1^{-/-} and corresponding littermate controls. FIG. 23A shows examples of fundus images of genotypes indicated. FIG. 23B shows a percentage distribution of pathologies seen at 12 months of age (GA and CNV: angiogenesis) in ^{rod}Tsc1^{-/-} S6K1^{-/-} and corresponding littermate controls.

FIG. 24 shows accumulation of ApoE and CFH, and loss of C3 expression at the RPE and BrM of 15 months old mice of genotypes indicated. Higher magnification of region between arrowheads is shown on top of each panel). (See text for details).

FIG. 25 shows percentage distribution of PE and PC di-DHA containing phospholipids in genotypes indicated. Measurements were performed in 2 months old mice (**P<0.01; ***P<0.001).

FIG. 26 shows percentage distribution of PE and PC di-DHA phospholipids in mice feed a DHA enriched diet from weaning onwards for 10 weeks. In mice with loss of TSC1 in rods DHA feeding did not the levels of di-DHA PE and PC lipids. Note: baseline levels between of ^{rod}Tsc1^{-/-} mice (FIG. 8) and of ^{rod}Tsc1^{-/-} S6K1^{-/-} mice (FIG. 25) differ slightly, which is likely due to the difference in the genetic background.

FIG. 27 shows p-S6 staining on retinal cross-sections of non-diseased and diseased individuals with AMD. There was a significant increase overall in retinas of AMD patients and in particular in the photoreceptor layer (P). The strongest staining was seen in the inner segment region. Photoreceptor segment region is marked with (S). The region marked with (S) includes inner segment, with the strongest p-S6 staining and part of the outer segment. Arrowheads point to a drusen deposit in this AMD patient. Each panel represents a different individual.

### DETAILED DESCRIPTION

Aspects of the disclosure relate to methods and compositions for treatment of certain ocular diseases and disorders, for example age-related macular degeneration (AMD). The disclosure is based, in part, on methods for treating AMD in a subject by administering one or more kinase inhibitors, for example one or more serine/threonine kinase inhibitors. In some embodiments, at least one of the serine/threonine kinase inhibitors is a mammalian target of rapamycin complex 1 (mTORC1) inhibitor. In some embodiments, at least one of the serine/threonine kinase inhibitors is a Ribosomal protein S6 kinase beta-1 (S6K1) inhibitor.

The mammalian Target of Rapamycin (mTOR) pathway has a vital role in the coordination of energy, nutrients and growth factor availability to regulate key biological processes including cellular growth, metabolism and protein synthesis through the phosphorylation of downstream ribosomal protein, S6 Kinase 1 (S6K1). mTOR modulates the activity of two important translational regulators, the ribosomal S6 kinases (S6K1 and S6K2), following changes in various cellular events (e.g., amino acid levels and energy sufficiency as well as stimulation by hormones and mitogens). These mTOR-regulated effectors (e.g., S6K1) control cell size and contribute to efficient G1 cell-cycle progression. Improper regulation of S6K1 contributes to carcinogenesis in cells with loss-of-function mutations in the tumor suppressors (e.g., PTEN, TSC1/2, or LKB) or upon gain-of-function mutations in many growth-factor receptors, phosphatidylinositol 3-kinase (PI3K), or Akt (protein kinase B). In addition, inappropriate mTOR signaling can contribute to metabolic diseases such as diabetes and obesity.

In some embodiments, mTOR initiates S6K1 activation in response to cellular energy status, nutrient levels, and mitogens. S6K1 activation is initiated by mTOR/raptor-mediated phosphorylation of T389, which requires the TOS motif located at the N terminus of S6K.

### Inhibitors

The disclosure relates in part to agents that inhibit expression or activity of one or more proteins in a mTORC1 pathway, for example mTORC1 or Ribosomal protein S6 kinase beta-1 (S6K1). Inhibitors of mTORC1 and/or S6K1 can be peptides, proteins, antibodies, small molecules, or nucleic acids.

As used herein the term “inhibitor” or “repressor” refers to any agent that inhibits, suppresses, represses, or decreases expression of a gene (e.g., reduces transcription or translation from a gene, such as MTOR, Raptor, MLST8, PRAS40, DEPTOR, RPS6KB1, etc.) or suppresses, represses, or decreases a specific activity, such as the activity of an mTORC1 protein and/or S6K1 protein. In some embodiments, an inhibitor selectively inhibits activity of mTORC1 or S6K1. As used herein, “selectively inhibits” refers to the inhibition of a specific target protein or gene (e.g., MTOR, RPS6KB1, mTOR protein, S6K protein, etc.) only and not inhibition of other genes or proteins. In some embodiments, an inhibitor is a direct inhibitor to S6K1 (e.g., an inhibitor that binds or interacts with S6K1 protein or nucleic acid encoding S6K1 that results in inhibition of S6K1 expression level and/or activity). In some embodiments, a direct S6K1 inhibitor is a peptide, protein, or an antibody directly binds and inhibits the activity of S6K1. In some embodiments, a direct S6K1 inhibitor is a small molecule inhibitor that directly binds and inhibits the activity of S6K1. In some embodiments, a direct S6K1 inhibitor is an inhibitory nucleic acid that directly binds S6K1 protein or S6K1 mRNA to inhibit the expression level and/or activity of S6K1.

mTORC1, also referred to as mammalian target of rapamycin complex 1 is a protein complex that comprises mTOR, regulatory-associated protein of mTOR (Raptor), mammalian lethal with SEC13 protein 8 (MLST8), PRAS40 and DEPTOR. In some embodiments, mTOR is encoded by an MTOR gene that comprises the sequence set forth in NCBI Reference Sequence number NM_004958.4. In some embodiments, an inhibitor binds directly to mTOR protein. In some embodiments, an inhibitor binds to a nucleic acid (e.g., a DNA, mRNA, etc.) encoding an mTOR protein.

Ribosomal protein S6 kinase beta-1 (S6K1), also known as p70S6 kinase (p70S6K, p70-S6K), is a protein kinase that in humans is encoded by the RPS6KB1 gene. In some embodiments, an inhibitor binds directly to S6K1 protein. In some embodiments, an inhibitor binds to a nucleic acid (e.g., a DNA, mRNA, etc.) encoding an S6K1 protein (e.g., a RPS6KB1 or mRNA encoded from such a gene). In some embodiments, a nucleic acid encoding S6K1 protein comprises the sequence set forth in NCBI Reference Sequence number NM_003161.4.

In some embodiments, an inhibitor when delivered to a cell results in a decrease in the level of expression and/or activity of a gene (e.g., MTOR, RPS6KB1, etc.) of at least 10%, 20%, 30%, 40%, 50%, 60%, 70%, 80%, 90%, 100%, 200% or 500% compared with the level of expression and/or activity of the gene in a control cell that has not been delivered an inhibitor. In some embodiments, delivery of an inhibitor to a cell results in a decrease in the level of expression and/or activity of gene(e.g., MTOR, RPS6KB1, etc.) in a range of 10% to 50%, 10% to 100%, 10% to 200%, 50% to 500% or more compared with the level of expression and/or activity of the gene in a control cell that has not been delivered an inhibitor. Methods of measuring gene expression and/or activity are known in the art and include, for example, quantitative PCR (qPCR), Western Blot, mass spectrometry (MS) assays, substrate assay, etc.

In some embodiments, an inhibitor (e.g., an inhibitor of mTOR or S6K1) is a small molecule. In some embodiments, the term “small molecule” refers to a synthetic or naturally occurring chemical compound, for instance a peptide or oligonucleotide that may optionally be derivatized, natural product or any other low molecular weight (often less than about 5 kilo Dalton) organic, bioinorganic or inorganic compound, of either natural or synthetic origin. Such small molecules may be a therapeutically deliverable substance or may be further derivatized to facilitate delivery. In some embodiments, an inhibitor inhibits S6K1 but not mTOR. In some embodiments, an inhibitor is a small molecule inhibitor of mTOR. Examples of mTOR inhibitors include but are not limited to rapamycin, everolimus, sirolimus, temsirolimus, deforolimus, KU-0063794, and salts, solvates, and analogues thereof. Examples of small molecule inhibitors of S6K1 include but are not limited to PF-4708671, rosmarinic acid methyl ester (RAME), A77 1726, and salts, solvates, and analogues thereof. In some embodiments, an inhibitor is a small molecule inhibitor of S6K1, for example, the S6K1 inhibitor as described in U.S. Pat. Nos. 10,144,726B2, 10,730,882B2, KR102106851B1, WO2016170163A1, WO2005019829A1, WO2005019829A1, each of which are incorporated herein by reference.

In some embodiments, an inhibitor is a protein. In some embodiments, the protein is a dominant negative variant of S6K1. In some embodiments, the dominant negative variant of S6K1 is S6K-DN, as described in Zhang et al. J Biol Chem. 2008 Dec. 19; 283(51): 35375-35382. In some embodiments, an inhibitor is a nucleic acid encoding the dominant negative variant of S6K1.

In some embodiments, an inhibitor is an antibody targeting S6K1. An antibody, as used herein, refers to a polypeptide that includes at least one immunoglobulin variable domain or at least one antigenic determinant, e.g., paratope that specifically binds to an antigen. In some embodiments, an antibody is a full-length antibody (e.g., anti-S6K1 antibody). In some embodiments, an antibody is a chimeric antibody (e.g., anti-S6K1 antibody). In some embodiments, an antibody is a humanized antibody (e.g., anti-S6K1 antibody). However, in some embodiments, an antibody is a Fab fragment, a Fab′ fragment, a F(ab′)2 fragment, a Fv fragment or a scFv fragment (e.g., a Fab fragment, a Fab′ fragment, a F(ab′)2 fragment, a Fv fragment or a scFv fragment targeting S6K1). In some embodiments, an antibody is a nanobody derived from a camelid antibody or a nanobody derived from shark antibody (e.g., anti-S6K1 nanobody). In some embodiments, an antibody is a diabody (e.g., anti-S6K1 diabody). In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. Non limiting examples of S6K1 antibody include antibody clones R.566.2, B12H16L8, B12HCLC, OTI6B2, etc.

In some embodiments, an inhibitor is an inhibitory oligonucleotide. Inhibitory oligonucleotides may interfere with gene expression, transcription and/or translation. Generally, inhibitory oligonucleotides bind to a target polynucleotide via a region of complementarity. For example, binding of inhibitory oligonucleotide to a target polynucleotide can trigger RNAi pathway-mediated degradation of the target polynucleotide (in the case of dsRNA, siRNA, shRNA, etc.), or can block the translational machinery (e.g., antisense oligonucleotides). In some embodiments, inhibitory oligonucleotides have a region of complementarity that is complementary with at least 8 (e.g., 8, 9, 10, 11, 12, 13, 14, 15, 16, 17, 18, 19, 20, 21, or more) nucleotides of an mRNA encoded by an MTOR gene or a RPS6KB1 gene. Inhibitory oligonucleotides can be single-stranded or double-stranded. In some embodiments, inhibitory oligonucleotides are DNA or RNA. In some embodiments, the inhibitory oligonucleotide is a hairpin-forming RNA selected from the group consisting of: antisense oligonucleotide, artificial miRNA (AmiRNA), siRNA, shRNA and miRNA. Generally, hairpin-forming RNAs are arranged into a self-complementary “stem-loop” structure that includes a single nucleic acid encoding a stem portion having a duplex comprising a sense strand (e.g., passenger strand) connected to an antisense strand (e.g., guide strand) by a loop sequence. The passenger strand and the guide strand share complementarity. In some embodiments, the passenger strand and guide strand share 100% complementarity. In some embodiments, the passenger strand and guide strand share at least 50%, at least 60%, at least 70%, at least 80%, at least 90%, at least 95%, or at least 99% complementarity. A passenger strand and a guide strand may lack complementarity due to a base-pair mismatch. In some embodiments, the passenger strand and guide strand of a hairpin-forming RNA have at least 1, at least 2, at least 3, at least 4, at least 5, at least 6, at least 7 at least 8, at least 9, or at least 10 mismatches. Generally, the first 2-8 nucleotides of the stem (relative to the loop) are referred to as “seed” residues and play an important role in target recognition and binding. The first residue of the stem (relative to the loop) is referred to as the “anchor” residue. In some embodiments, hairpin-forming RNA have a mismatch at the anchor residue. Hairpin-forming RNAs are useful for translational repression and/or gene silencing via the RNAi pathway. Due to having a common secondary structure, hairpin-forming RNAs share the characteristic of being processed by the proteins Drosha and Dicer prior to being loaded into the RNA-induced silencing complex (RISC). Duplex length amongst hairpin-forming RNAs can vary. In some embodiments, a duplex is between about 19 nucleotides and about 200 nucleotides in length. In some embodiments, a duplex is between about between about 14 nucleotides to about 35 nucleotides in length. In some embodiments, a duplex is between about 19 and 150 nucleotides in length. In some embodiments, hairpin-forming RNA has a duplex region that is 19, 20, 21, 22, 23, 24, 25, 26, 27, 28, 29, 30, 31, 32, or 33 nucleotides in length. In some embodiments, a duplex is between about 19 nucleotides and 33 nucleotides in length. In some embodiments, a duplex is between about 40 nucleotides and 100 nucleotides in length. In some embodiments, a duplex is between about 60 and about 80 nucleotides in length.

In some embodiments, the hairpin-forming RNA targeting S6K1 is an artificial microRNA (AmiRNA). As used herein “artificial miRNA” or “amiRNA” refers to an endogenous pri-miRNA or pre-miRNA (e.g., a miRNA backbone, which is a precursor miRNA capable of producing a functional mature miRNA), in which the miRNA and miRNA* (e.g., passenger strand of the miRNA duplex) sequences have been replaced with corresponding amiRNA/amiRNA* sequences that direct highly efficient RNA silencing of the targeted gene, for example as described by Eamens et al. (2014), Methods Mol. Biol. 1062:211-224. In some embodiments, the AmiRNA backbone is derived from a pri-miRNA selected from the group consisting of pri-MIR-21, pri-MIR-22, pri-MIR-26a, pri-MIR-30a, pri-MIR-33, pri-MIR-64, pri-MIR-122, pri-MIR-155, pri-MIR-375, pri-MIR-199, pri-MIR-99, pri-MIR-194, pri-MIR-155, and pri-MIR-451.

In some embodiments, an inhibitory nucleic acid targeting S6K1 include any inhibitory nucleic acid known in the art, for example, an inhibitory nucleic acid targeting S6K2 as described in US20030083284, and US20070191259A1, each of which is incorporated herein by reference.

In some embodiments, inhibitory oligonucleotides are modified nucleic acids. The term “nucleotide analog” or “altered nucleotide” or “modified nucleotide” refers to a non-standard nucleotide, including non-naturally occurring ribonucleotides or deoxyribonucleotides. In some embodiments, nucleotide analogs are modified at any position so as to alter certain chemical properties of the nucleotide yet retain the ability of the nucleotide analog to perform its intended function. Examples of positions of the nucleotide which may be derivitized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10(4):297-310.

Nucleotide analogs may also comprise modifications to the sugar portion of the nucleotides. For example the 2′ OH-group may be replaced by a group selected from H, OR, R, F, Cl, Br, I, SH, SR, NH2, NHR, NR2, COOR, or, wherein R is substituted or unsubstituted C.sub.1-C.sub.6 alkyl, alkenyl, alkynyl, aryl, etc. Other possible modifications include those described in U.S. Pat. Nos. 5,858,988, and 6,291,438. A locked nucleic acid (LNA), often referred to as inaccessible RNA, is a modified RNA nucleotide. The ribose moiety of an LNA nucleotide is modified with an extra bridge connecting the 2′ oxygen and 4′ carbon.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) preferably decrease the rate of hydrolysis of, for example, polynucleotides comprising said analogs in vivo or in vitro. In some embodiments, the inhibitory oligonucleotide is a modified inhibitory oligonucleotide. In some embodiments, the modified inhibitory oligonucleotide comprises a locked nucleic acid (LNA), phosphorothioate backbone , and/or a 2′-O-Me modification.

### Methods

Aspects of the disclosure relate to methods of inhibiting drusen formation in an ocular tissue, the method comprising administering to cells of the ocular tissue one or more inhibitors of mammalian target of rapamycin complex 1 (mTORC1), for example MTOR or RPS6KB1 (or a protein encoded by such genes). In some embodiments, the cell is in vitro. In some embodiments, the cell is in a subject (e.g., the cell is in vivo).

In some embodiments, the disclosure provides a method for treating age-related macular degeneration (AMD) in a subject, the method comprising administering to the subject one or more inhibitors of mTORC1 (e.g., MTOR or RPS6KB1 or a protein encoded by such genes).

Age-related Macular Degeneration (AMD) is one of the leading causes for visual impairment in the elderly. The disease is multi-factorial including genetic and non-genetic risk factors. Among the non-genetic risk factors smoking and diet have been shown to be the most important modifiable risk factors. Omega-3 fatty acid rich foods, in particular Docosahexaenoic acid (DHA) rich foods, have been found to reduce disease risk. Similarly, high DHA plasma levels correlate with reduced disease risk. Moreover, individuals with AMD have a 30% reduction in retinal DHA levels.

As used herein, a “subject” is interchangeable with a “subject in need thereof”, both of which may refer to a subject having age-related macular degeneration (AMD), or a subject having an increased risk of developing such a disorder relative to the population at large (e.g., a subject having one or more genetic mutations associated with AMD, for example complement factor H (CFH), etc.). A subject in need thereof may be a subject exhibiting one or more signs or symptoms of AMD. In some embodiments, a subject (e.g., a subject has or at increased risk of having AMD) has or is at an increased risk of over-activation of S6K1 (e.g., constitutive activation of S6K1) as compared to a subject not at risk. In some embodiments, loss of TSC1 and/or TSC2 (e.g., loss of expression or function of TSC1 and/or TSC2) leads to over-activation of S6K1. In some embodiments, a subject with over-activation of S6K1 is TSC1 deficient (e.g., loss of expression or function of TSC1). In some embodiments, a subject with over-activation of S6K1 is TSC2 deficient (e.g., loss of expression or function of TSC2). In some embodiments, a subject with over-activation of S6K1 is TSC1 and TSC2 deficient (e.g., loss of expression or function of TSC1 and/or TSC2). A subject can be a human, non-human primate, rat, mouse, cat, dog, or other mammal.

As used herein, the terms “treatment”, “treating”, and “therapy” refer to therapeutic treatment and prophylactic or preventative manipulations. The terms further include ameliorating existing symptoms, preventing additional symptoms, ameliorating or preventing the underlying causes of symptoms, preventing or reversing causes of symptoms, for example, symptoms associated with age-related macular degeneration (AMD). Thus, the terms denote that a beneficial result has been conferred on a subject with a disorder (e.g., AMD), or with the potential to develop such a disorder. Furthermore, the term “treatment” is defined as the application or administration of an agent (e.g., therapeutic agent or a therapeutic composition) to a subject, or an isolated tissue or cell line from a subject, who may have a disease, a symptom of disease or a predisposition toward a disease, with the purpose to cure, heal, alleviate, relieve, alter, remedy, ameliorate, improve or affect the disease, the symptoms of disease or the predisposition toward disease. “Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease (e.g., AMD).

The disclosure is based, in some aspects, on methods of treating AMD which comprise administering to the subject di-docosahexaenoic acid (DHA) in addition to one or more inhibitors. In some embodiments, the DHA is administered as a dietary supplement (e.g., administered orally).

Therapeutic agents or therapeutic compositions may include a compound in a pharmaceutically acceptable form that prevents and/or reduces the symptoms of a particular disease (e.g., AMD). For example a therapeutic composition may be a pharmaceutical composition that prevents and/or reduces the symptoms of AMD. It is contemplated that the therapeutic composition of the present invention will be provided in any suitable form. The form of the therapeutic composition will depend on a number of factors, including the mode of administration as described herein. The therapeutic composition may contain diluents, adjuvants and excipients, among other ingredients as described herein.

The pharmaceutical compositions containing an inhibitor and/or other compounds can be administered by any suitable route for administering medications. A variety of administration routes are available. The particular mode selected will depend, of course, upon the particular agent or agents selected, the particular condition being treated, and the dosage required for therapeutic efficacy. The methods of this disclosure, generally speaking, may be practiced using any mode of administration that is medically acceptable, meaning any mode that produces therapeutic effect without causing clinically unacceptable adverse effects. Various modes of administration are discussed herein. For use in therapy, an effective amount of the inhibitor and/or other therapeutic agent can be administered to a subject by any mode that delivers the agent to the desired surface, e.g., mucosal, systemic.

In some embodiments, an inhibitory oligonucleotide can be delivered to the cells via an expression vector engineered to express the inhibitor oligonucleotide. An expression vector is one into which a desired sequence may be inserted, e.g., by restriction and ligation, such that it is operably joined to regulatory sequences and may be expressed as an RNA transcript. An expression vector typically contains an insert that is a coding sequence for a protein or for a inhibitory oligonucleotide such as an shRNA, a miRNA, or an miRNA. Vectors may further contain one or more marker sequences suitable for use in the identification of cells that have or have not been transformed or transfected with the vector. Markers include, for example, genes encoding proteins that increase or decrease either resistance or sensitivity to antibiotics or other compounds, genes that encode enzymes whose activities are detectable by standard assays or fluorescent proteins, etc.

As used herein, a coding sequence (e.g., protein coding sequence, miRNA sequence, shRNA sequence) and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide. It will be appreciated that a coding sequence may encode an miRNA, shRNA or miRNA.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Such 5′ non-transcribed regulatory sequences will include a promoter region that includes a promoter sequence for transcriptional control of the operably joined gene. Regulatory sequences may also include enhancer sequences or upstream activator sequences as desired. The vectors of the disclosure may optionally include 5′ leader or signal sequences.

In some embodiments, a virus vector for delivering a nucleic acid molecule is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses, a modified retrovirus, a nonreplicating retrovirus, a replication defective Semliki Forest virus, canarypox virus and highly attenuated vaccinia virus derivative, non-replicative vaccinia virus, replicative vaccinia virus, Venzuelan equine encephalitis virus, Sindbis virus, lentiviral vectors and Ty virus-like particle. Another virus useful for certain applications is the adeno-associated virus. The adeno-associated virus is capable of infecting a wide range of cell types and species and can be engineered to be replication-deficient. It further has advantages, such as heat and lipid solvent stability, high transduction frequencies in cells of diverse lineages, including hematopoietic cells, and lack of superinfection inhibition thus allowing multiple series of transductions. The adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

In general, other useful viral vectors are based on non-cytopathic eukaryotic viruses in which non-essential genes have been replaced with the gene of interest. Non-cytopathic viruses include certain retroviruses, the life cycle of which involves reverse transcription of genomic viral RNA into DNA with subsequent proviral integration into host cellular DNA. In general, the retroviruses are replication-deficient (e.g., capable of directing synthesis of the desired transcripts, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W. H. Freeman Co., New York (1990) and Murry, E. J. Ed. “Methods in Molecular Biology,” vol. 7, Humana Press, Inc., Clifton, N.J. (1991).

Various techniques may be employed for introducing nucleic acid molecules of the disclosure into cells, depending on whether the nucleic acid molecules are introduced in vitro or in vivo in a host. Such techniques include transfection of nucleic acid molecule-calcium phosphate precipitates, transfection of nucleic acid molecules associated with DEAE, transfection or infection with the foregoing viruses including the nucleic acid molecule of interest, liposome-mediated transfection, and the like. Other examples include: N-TER™ Nanoparticle Transfection System by Sigma-Aldrich, FECTOFLY™ transfection reagents for insect cells by Polyplus Transfection, Polyethylenimine “Max” by Polysciences, Inc., Unique, Non-Viral Transfection Tool by Cosmo Bio Co., Ltd., LIPOFECTAMINE™ LTX Transfection Reagent by Invitrogen, SATISFECTION™ Transfection Reagent by Stratagene, LIPOFECTAMINE™ Transfection Reagent by Invitrogen, FUGENE® HD Transfection Reagent by Roche Applied Science, GMP compliant IN VIVO-JETPEI™ transfection reagent by Polyplus Transfection, and Insect GENEJUICE® Transfection Reagent by Novagen.

Delivery of a S6K1 inhibitor (e.g., any one of the S6K1 inhibitor described herein or a combination thereof) to a mammalian subject may be by, for example, intramuscular injection or by administration into the bloodstream of the mammalian subject. Administration into the bloodstream may be by injection into a vein, an artery, or any other vascular conduit. In some embodiments, a S6K1 inhibitor (e.g., any one of the S6K1 inhibitor described herein or a combination thereof) are administered into the bloodstream by way of isolated limb perfusion, a technique well known in the surgical arts, the method essentially enabling the artisan to isolate a limb from the systemic circulation prior to administration of a S6K1 inhibitor (e.g., any one of the S6K1 inhibitor described herein or a combination thereof). Moreover, in certain instances, it may be desirable to deliver a S6K1 inhibitor (e.g., any one of the S6K1 inhibitor described herein or a combination thereof) to the ocular tissue of a subject. An S6K1 inhibitor (e.g., any one of the S6K1 inhibitor described herein or a combination thereof) may be delivered directly to the eye by injection into, e.g., subretinal or intravitreal administration. In some embodiments, a S6K1 inhibitor (e.g., any one of the S6K1 inhibitor described herein or a combination thereof) as described in the disclosure are administered by intravenous injection. In some embodiments, a S6K1 inhibitor (e.g., any one of the S6K1 inhibitor described herein or a combination thereof) are administered by intrathecal injection. In some embodiments, a S6K1 inhibitor (e.g., any one of the S6K1 inhibitor described herein or a combination thereof) are delivered by intramuscular injection.

Aspects of the instant disclosure relate to compositions comprising a S6K1 inhibitor (e.g., any one of the S6K1 inhibitor described herein or a combination thereof). In some embodiments, a composition further comprises a pharmaceutically acceptable carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

The compositions of the disclosure may comprise one S6K1 inhibitor alone (e.g., siRNA targeting S6K1), or in combination with one or more other S6K1 inhibitors (e.g., an S6K1 antibody or a polypeptide targeting S6K1). In some embodiments, a composition comprises 1, 2, 3, 4, 5, 6, 7, 8, 9, 10, or more different S6K1 inhibitors.

Suitable carriers may be readily selected by one of skill in the art in view of the indication for which the S6K1 inhibitor (e.g., any one of the S6K1 inhibitor described herein or a combination thereof) is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the S6K1 inhibitor and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, parachlorophenol, and poloxamers (non-ionic surfactants) such as Pluronic® F-68. Suitable chemical stabilizers include gelatin and albumin.

The S6K1 inhibitor or the composition thereof is administered in sufficient amounts to provide the cells of a desired tissue (e.g., ocular tissue) sufficient levels to inhibit S6K1 without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, oral administration, and other parental routes of administration. Routes of administration may be combined, if desired.

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver a S6K1 inhibitor (e.g., any one of the S6K1 inhibitor described herein or a combination thereof) in suitably formulated pharmaceutical compositions disclosed herein either subretinally, intravitreally, subcutaneously, intraopancreatically, intranasally, parenterally, intravenously, intramuscularly, intrathecally, or orally, intraperitoneally, or by inhalation.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the S6K1 inhibitor in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The S6K1 compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

Delivery vehicles such as liposomes, nanocapsules, microparticles, microspheres, lipid particles, vesicles, and the like, may be used for the introduction of the compositions of the present disclosure into suitable host cells. In particular, the S6K1 inhibitor may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the S6K1 inhibitor disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the S6K1 inhibitor may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

### EXAMPLES

### Example 1

Activation of mTORC1 in human photoreceptors (PRs) is an adaptive response to the nutrient shortage photoreceptors experience during the early disease process. Increased expression of aerobic glycolysis genes in photoreceptors of human AMD samples has been observed, suggesting that mTORC1 activity is increased in humans having AMD.

This Example describes in vivo experiments performed on a mouse model of age-related macular degeneration (AMD). A mouse model of AMD was produced by increasing expression of aerobic glycolysis genes by genetic engineering. Briefly, mammalian target of rapamycin 1 (mTORC1) activity was increased in mice by deleting the Tuberous sclerosis complex (TSC1). The resulting mice, referred to as ^{rod}TSC1^{-/-}. include both early (e.g., “wet AMD”) pathologies, including accumulation of apolipoprotein E (ApoE) and complement factor H (CHF), and late (e.g., “dry AMD”) pathologies, including neovascularization and geographic atrophy (GA) of the RPE and underlying photoreceptors.

In addition, these mice show also a reduction di-DHA lipids in phosphatidylethanolamine and phosphatidylcholine. Coincidently, DHA rich food has been shown to reduce the risk for disease progression. Data indicate that it was not the increase in aerobic glycolysis per se, but rather the gene expression changes that accompany the increase in mTORC1 activity that cause AMD. For example, the reduction in di-DHA phospholipids is due, in some embodiments, to a reduction in expression of the enzyme(s) that are responsible for the synthesis.

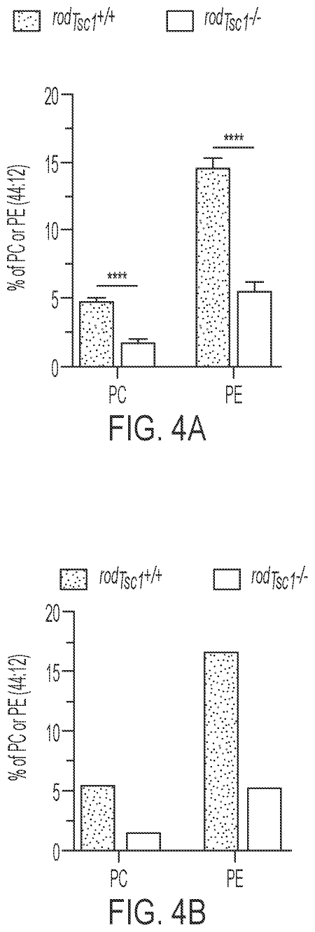

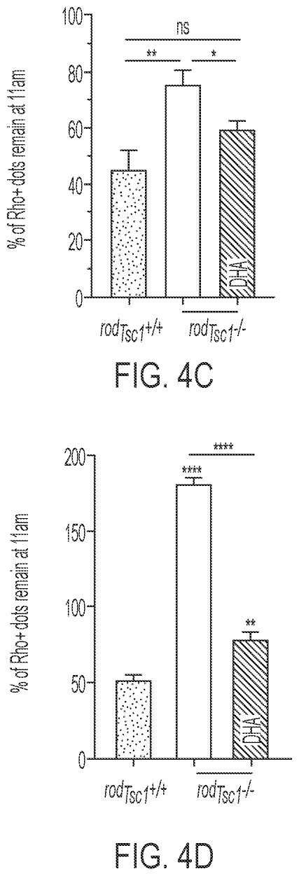

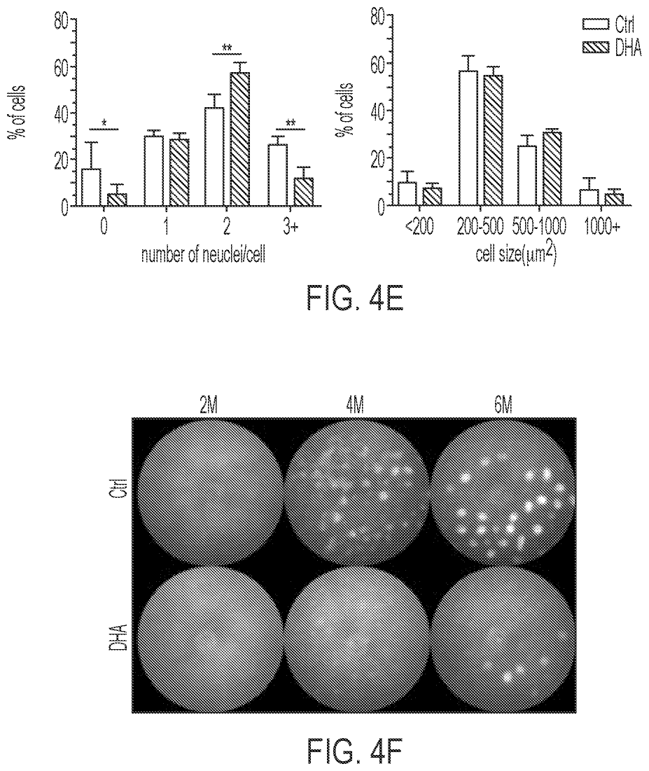

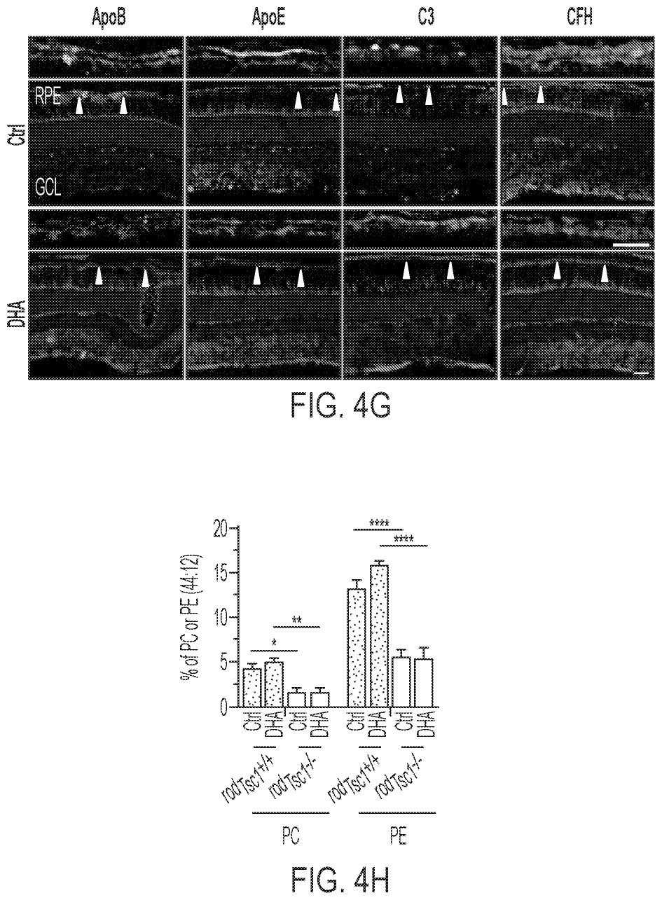

Mice with activated mTORC1 in PRs also displayed other early disease features such as a delay in photoreceptor outer segments (POS) clearance, accumulation of lipofuscin in the retinal-pigmented epithelium (RPE) and of lipoproteins at the Bruch's membrane (BrM), as well as changes in complement accumulation. POSs are rich in lipids and mTORC1 is known to regulate lipid synthesis. To determine a cause for the delayed POS clearance by the RPE, the retinal lipid composition of ^{rod}Tsc1^{-/-} mice was profiled. A ˜3-fold decrease in di-DHA (44:12) containing phosphatidylethanolamine (PE) and phosphatidylcholine (PC) lipids in total retinal (FIG. 4A) and POS preparations (FIG. 4B) were observed. To test if this drop in di-DHA PE and PC lipids contributes to the delay in POS clearance ^{rod}Tsc1^{-/-} mice were fed a diet enriched with 2% DHA. Feeding ^{rod}Tsc1^{-/-} mice a 2% DHA enriched diet from weaning onwards improved POS clearance at 2M (FIG. 4C). To test if delayed POS clearance can also be improved once the delay has occurred, 6M old ^{rod}Tsc1^{-/-} mice were fed the DHA enriched diet for 2 weeks. This had an even more pronounced effect, as POS clearance was more affected at 6M (FIG. 4D). To determine if dietary DHA also affected overall RPE health, mice were kept on the DHA diet from weaning onwards until 6M. This reduced the percentage of polynucleated RPE cells (FIG. 4E), improved fundus pathologies (FIG. 4F), prevented the accumulation of ApoB, ApoE and CFH, and restored C3 expression (FIG. 4G). Differences in RPE hypertrophy were not evident. None of 12 DHA-fed mice (n=12) developed any GA by 6M, while 1 out of 6 mice on the control diet did. Re-profiling of the retinal lipids after 10 weeks of DHA feeding indicated that levels of di-DHA containing PE and PC lipids were not restored. This indicates that DHA acted directly on the RPE to improve overall PRE health (FIG. 4H). In all, the data indicate that activated mTORC1 in rods affects the retinal lipid composition, which affects overall RPE health.

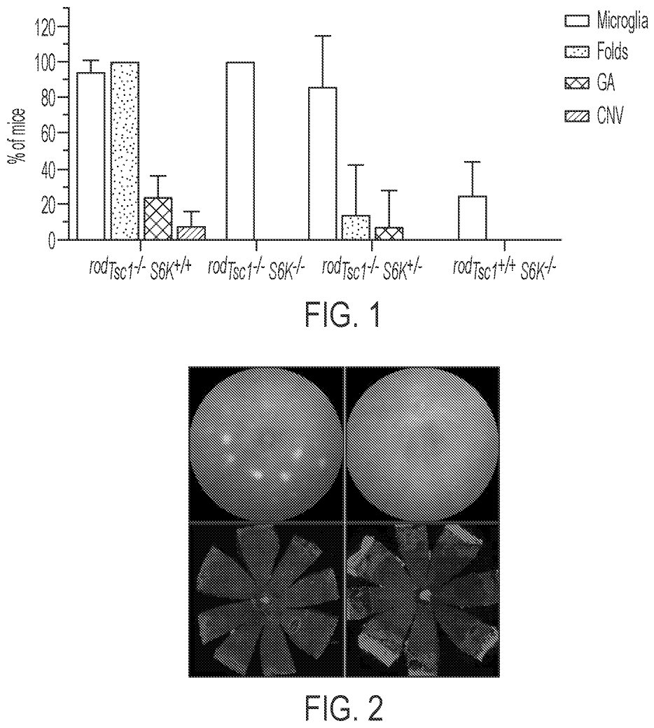

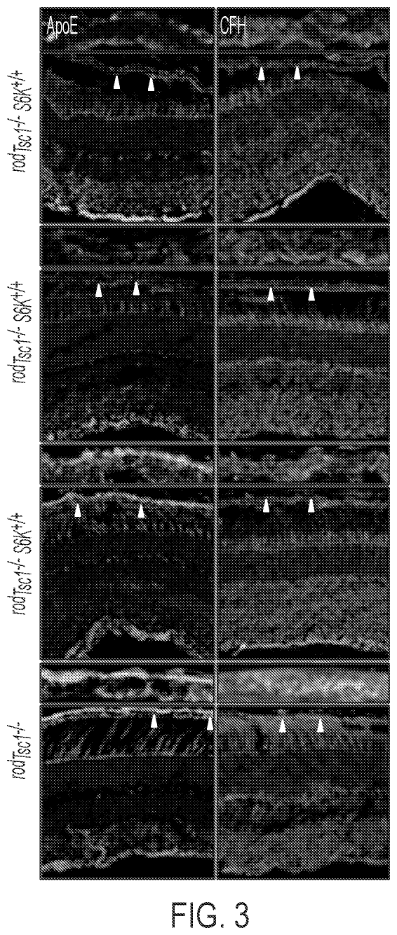

Additional mouse models, such as mice with activated mTORC1 and loss of S6K1, were produced to investigate the effects of ribosomal protein S6 kinase beta-1 (S6K1, also referred to as p70S6 kinase) function on development of AMD pathologies. These mice did not develop advanced AMD pathologies. FIG. 1 shows pathology distribution in mice with loss of TSC1 in rods and two normal copies of S6K1 (^{rod}TSC1^{-/-} S6K1^{+/+}), with loss of TSC1 in rods and loss of S6K1 (^{rod}TSC1^{-/-} S6K1^{-/-}), with loss of TSC1 in rods and loss of one copy of S6K1 (^{rod}TSC1^{-/-} S6K1^{-/+}), and with two normal copies of TSC1 and complete loss of S6K1 (^{rod}TSC1^{+/+} S6K1^{-/-}). Complete loss of S6K1 in the context of loss of TSC1 in rods prevents advanced AMD pathologies. FIG. 2 shows fundus images and retinal-pigmented epithelium flat mounts showing that mice with one copy of S6K1 in and loss of TSC1 (^{rod}TSC1^{-/-} S6K1^{-/+}) develop fundus pathologies (left) and GA as seen on flat mounts. In contrast, pathology was not observed in mice with loss of both TSC1 and S6K1 (^{rod}TSC1^{-/-} S6K1^{-/-}). FIG. 3 shows deletion of S6K1 in the context of loss of TSC1 prevents accumulation of ApoE and complement factor H (CHF), which are both hallmarks of early-stage AMD.

These data indicate that, in the context of increased mTORC1 activity, inhibition of S6K1 prevents occurrence of both early and late AMD-related pathologies.

### Example 2

### Human Tissue Samples

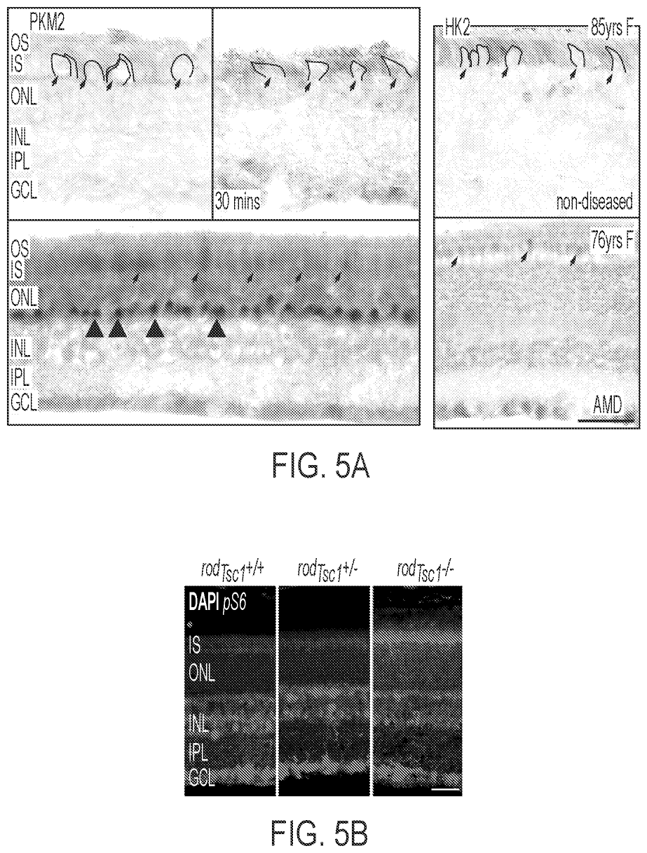

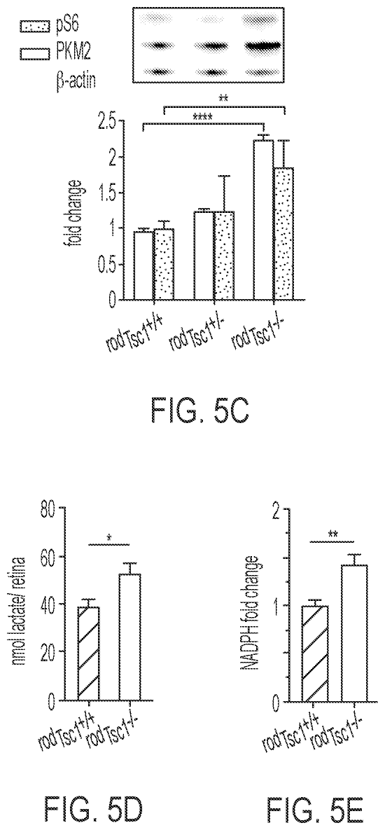

Age and sex of human postmortem eye samples are indicated in FIG. 5A, and FIGS. 11A-11B. All staining on human tissue sample used cryopreserved tissue sections.

### Animals

The conditional Tsc1 and Raptor alleles as well as the rod iCre-75 and cone-Cre have all been previously described. All mice were genotyped for the absence of the rd8 mutation. Mice were kept on a 12 hr-light/12 hr-dark cycle with unrestricted diets. Equal numbers of male and female mice were used in all experiments. No sex-specific differences were noted. The DHA diet was made by replacing 2% of soybean oil in the AIN-93G lab diet from Dyets, Inc., with 2% DHASCO from DSM. The AIN-93G diet was used as a control diet for all DHA experiments. Except for the DHA and DHA control experiments, all animals were kept on a control diet; AIN-93G control diet and the 5P75* facility diet differ in their soybean oil content, which are 7% and 5%, respectively.

### Funduscopy and Angiography

Funduscopy was performed. Ages and number of mice analyzed for a given experiment are indicated in figures and/or legends. Angiography was performed immediately following funduscopy imaging by injecting 125 mg/kg of a fluorescein sodium solution subcutaneously behind the neck. Images were acquired with the Micron III from Phoenix Technology Group. Overall accuracy of GA diagnosis by funduscopy was confirmed on RPE flat mounts of 22 eyes, 7 of which were diagnosed with GA by funduscopy. Of the 22 eyes, 9 were confirmed on RPE flat mounts to have GA.

### Optical Coherence Tomography (OCT)

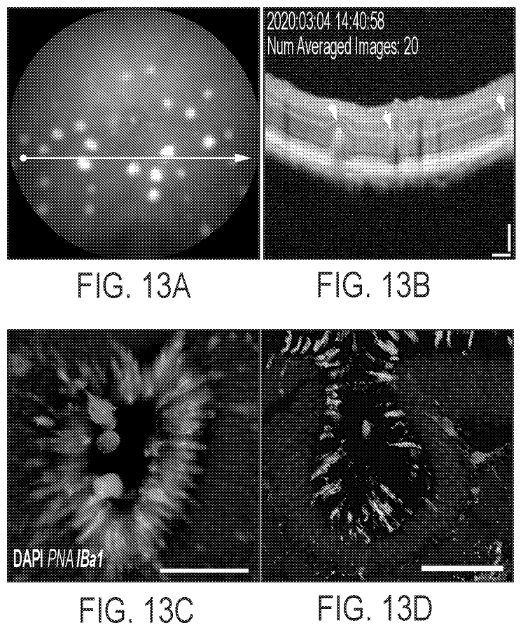

OCT was performed with a system from Bioptigen (Model: 70-20000). OCT in FIG. 13 was acquired during manuscript revision with a new Micron IV system from Phoenix Technology Group. Mice were anesthetized with a mixture of ketamine/xylazine (100 mg/kg and 10 mg/kg). One drop of both Phenylephrine (2.5%) and Tropicamide (1%) was applied for pupil dilation 10 min prior to recording. After the recording mice were allowed to recover on a warm heating tray.

### Electroretinography (ERG) Analysis

ERGs were performed with the Celeris system for scotopic, photopic and C-wave ERGs. Number of mice per group is indicated in the Figure legends. Mice were not pre-screened for their eye pathologies.

### Lactate Assay

Lactate assay (L-Lactate Assay kit, Abcam, Cat# ab65330) was performed with 2-month-old mice using four biological samples, each composed of both retinas from the same animal. Each biological measurement was performed in triplicate. Retinas were dissected in ice cold PBS and processed according to manufacturer's instructions.

### NADPH Assay

NADPH assay (NADP/NADPH Assay Kit, Sigma, Cat# MAK312) was performed with 2-month-old mice using 7-8 biological samples, each composed of one retina. Each biological measurement was performed in duplicate. Retinas were dissected in ice cold PBS and processed according to manufacturer's instructions.

Quantitative Western blot Analyses

All Western blot quantifications used three biological samples with each sample consisting of both retinas from the same mouse. The analysis of each sample was performed in triplicate. Proteins were extracted as follows: enucleated eyes were dissected in cold PBS buffer. Dissected retinas were immediately transferred into RIPA buffer (Thermo Scientific, cat# 89900) with protease & phosphatase inhibitors (1:100 dilution; cat#1861281) and homogenized by sonication. After 10 min centrifugation at 4° C. at 13000 RPM, protein extracts were transferred into a fresh tube and protein concentration was quantified with the Bio-Rad Protein Assay (cat# 500-0113,0114,0115). To quantify PKM2 and p-S6 expression levels, 5 μg and 10 μg of total protein, respectively, were loaded. The following primary antibodies from Cell Signaling Technology were used: rabbit anti-PKM2 antibody (1:4,000; Cat#4053), rabbit anti-pS6 (Ser240/244) (1:1000; Cat#5364), and for normalization mouse anti-β-actin antibody (1:1,000, Cat#3700). Protein detection was done using fluorescently labeled secondary (1:10,000) antibodies from Licor in combination with the Odyssey system. Quantification was performed with Image Studio software.

### Immunohistochemistry

Immunohistochemistry (IHC) and immunofluorescence on either cryo-preserved sections (10 μm thickness) or RPE/retina whole mounts were performed. The following primary antibodies were used: rabbit anti-PKM2 (1:1000; Cell Signaling Technology, Cat#4053), rabbit anti-ZO1 (1:100; Invitrogen, Cat#40-2200), and rabbit anti-Iba1 (1:300; Wako, Cat#019-19741), mouse anti-CRE-Recombinase (1:500, Covance, Cat#PRB-106P), mouse anti-Rhodopsin (1:100, originally obtained from the University of British Columbia, Clone 1D4, available from Abcam, cat# 5417) all diluted in PBS with 0.3% Triton X-100 and 5% bovine serum albumin (BSA, Cell Signaling Technology). For the rabbit anti-pS6 (Ser240/244) antibody (1:300; Cell Signaling Technology, Cat# 5364), PBS was replaced with TBS. For the rabbit anti-Apolipoprotein B (ApoB) (1:800; Abcam, Cat# 20737), goat anti-Apolipoprotein E (ApoE) (1:1,000, Millipore, Cat#178479), rabbit anti-CFH (1:300; Cat# ABIN3023097) and goat anti-mouse complement C3 (1:300; MP Biomedicals, cat# 55510), Triton X-100 was replaced with 0.2% Saponin. The following reagents already had a chromophore conjugated: rhodamine phalloidin (1:1,000; Life Technologies, Cat# R415), fluorescein peanut agglutinin lectin (PNA) (1:1,000; Vector Laboratories, Cat# FL1071) and fluorescein Griffonia Simplicifonia Lectin I (GSL I) isolectin B4 (1:300; Vector Laboratories, Cat# FL-1201). Nuclei were counterstained with 4′, 6-diamidino-2-phenylindole (DAPI) (Sigma-Aldrich, Cat# 9542). All secondary antibodies (1:500, donkey) were purchased from Jackson Immuno Research and were purified F(ab)2 fragments that displayed minimal cross-reactivity with other species. An exception of this was the immunohistochemistry staining, which used the ImmPACT VIP kit (Vector Laboratories, Cat# SK-4605). Expression changes for ApoB, ApoE, C3 and CFH were confirmed in at least 3 individual animal per genotype. All images were visualized with a Leica DM6 Thunder microscope with a 16 bit monochrome camera.

### RPE Polynucleation and Cell Size Quantification

RPE whole mounts were collected and stained with anti-ZO1 antibody by immunofluorescence in order to highlight RPE cell boundaries. For quantification, 10 images of 22,500 μm² each were selected within a radius of 1.5 mm from the center. Because the distribution of affected regions can be random in control and experimental mice, the 10 most affected areas within one RPE flat mount were selected, avoiding regions of GA in experimental mice. Images for quantification were acquired at 20X. IMARIS software was used to quantify the number of nuclei and cell area of each RPE cell within a given image. Each image had 30-50 RPE cells, meaning per RPE flat mount we analyzed 300-500 RPE cells to calculate the average number of nuclei per RPE cell and the average RPE cell size. Each experimental group consisted of 6-8 RPE flat mounts. The age and number of RPE flat mounts per group is indicated in the corresponding figure legend.

### Analysis of POS Clearance by the RPE

Quantification of POS clearance was performed: Per RPE flat mount, 10 areas of 40,000 μm² within a 1.5 mm radius from the center were selected randomly to quantify the number of RHODOPSIN positive dots per RPE cell. Images for quantification were acquired at 20X. RPE cell boundaries were detected with anti-ZO1 antibody. Quantification was performed using IMARIS imaging processor by selecting a dot diameter >2 μm to count dots and by counting the number of RPE cells per imaged field. The average dot number per RPE cell for a given RPE flat mount was obtained by averaging the results of the 10 fields. This number was then used to generate the average of the biological replicates, as indicated in the individual figures, per genotype and time point. All POS clearance experiments were performed with 2M-old mice except for 6M-old mice that were fed the DHA-enriched diet for 2 weeks.

### Quantification of Rod Survival

Quantification of rod survival was performed. Each group used 6 retinas per quantification. Retinal sections were cut in a dorsal to ventral direction. TUNEL assay. TUNEL assay (Roche, Cat# 12156792910) was performed according to manufacturer's instructions. After the TUNEL reaction, tissue was processed for immunofluorescence staining as described above. Semithin and transmission electron microscopy (EM) were performed.

### Lipid Profiling

Each biological sample consists of two retinas from the same animal. The following numbers of biological samples were used: ^{rod}Tsc1^{-/-}=9; ^{rod}Tsc1^{+/+}=6; ^{cone}Tsc1^{-/-}=6; ^{cone&rod}Tsc1^{-/-}=3, and the DHA experiments used 3 biological samples per condition. The POS preparations pooled 6 retinas from 3 animals per genotype. Briefly, tissue was homogenized in 40% aqueous methanol and then diluted to a concentration of 1:40 with 2-propanol/methanol/chloroform (4:2:1 v/v/vol) containing 20 mM ammonium formate and 1.0 μM PC (14:0/14:0), 1.0 μM PE (14:0/14:0), and 0.33 μM PS (14:0/14:0) as internal standards. Samples were introduced into a triple-quadrupole mass spectrometer (TSQ Ultra, Thermo Scientific) by using a chip-based nano-ESI source (Advion NanoMate) operating in infusion mode. PC lipids were measured using precursor ion scanning of m/z 184, PE lipids were measured using neutral loss scanning of m/z 141, and PS lipids were measured using neutral loss scanning of m/z 185. All species detected for each group are represented as a relative percentage of the sum based on their response values. Abundances of lipid molecular species were calculated using the Lipid Mass Spectrum Analysis (LIMSA) software (University of Helsinki, Helsinki, Finland).

### Statistical Analysis

Multiple t-test was used for two-group comparisons and two-way ANOVA for comparisons of more than two groups. Both analysis types were two-tailed. Significance levels: *p<0.05; **p<0.01; ***p<0.001; ****p<0.0001. All bar graphs indicate mean and error bars represent the S.E.M. Fundus analysis bar graphs show the percentage of mice that developed the retinal pathologies described while error bars represent margin of errors calculated with 90% confidence.

### HK2 and PKM2 Expression are Increased in PRs of AMD Patients

To determine whether PR metabolism differs in individuals with AMD, the expression of these two key metabolic genes were investigated in human donor eyes with or without AMD. On retinal sections, increased expression of PKM2 and HK2 in PRs of AMD patients (n=3) was observed, with the highest increase found in cones (FIG. 5A and FIGS. 11A-11B). Expression in non-diseased retinas was low for PKM2, as those sections required up to 5X longer exposure to the histochemical reagent in order for a strong signal to emerge (FIG. 5A). To allow for a more linear comparison between samples, the experiments were repeated using immunofluorescence (FIG. 11A). A 2-fold scaling of the signal between non-diseased and diseased tissue was sufficient to reveal a PR signal in non-diseased tissue without causing overexposure of the signal in diseased retinas. Expression of both genes in mouse has been observed to decline with age (FIG. 11C). Dta indicate that levels HK2 and PKM2 increase in PRs of individuals with AMD, indicating that glucose availability is reduced in diseased individuals.

^{rod}Tsc1^{-/-} Mice Develop Advanced AMD Pathologies

To determine the effect of metabolic changes on retinal and RPE health in wild-type mice, mTORC1 was constitutively activated in rods by deletion of the Tsc1 gene (henceforth referred to as ^{rod}Tsc1^{-/-}) using the Cre-lox system. mTORC1 activity was confirmed by immunofluorescence and Western blot analyses for phosphorylated ribosomal protein S6 (p-S6) (FIGS. 5B-5C). Similarly, changes in PR metabolism were confirmed by quantifying retinal PKM2, lactate and NADPH levels (FIGS. 5C-5E).

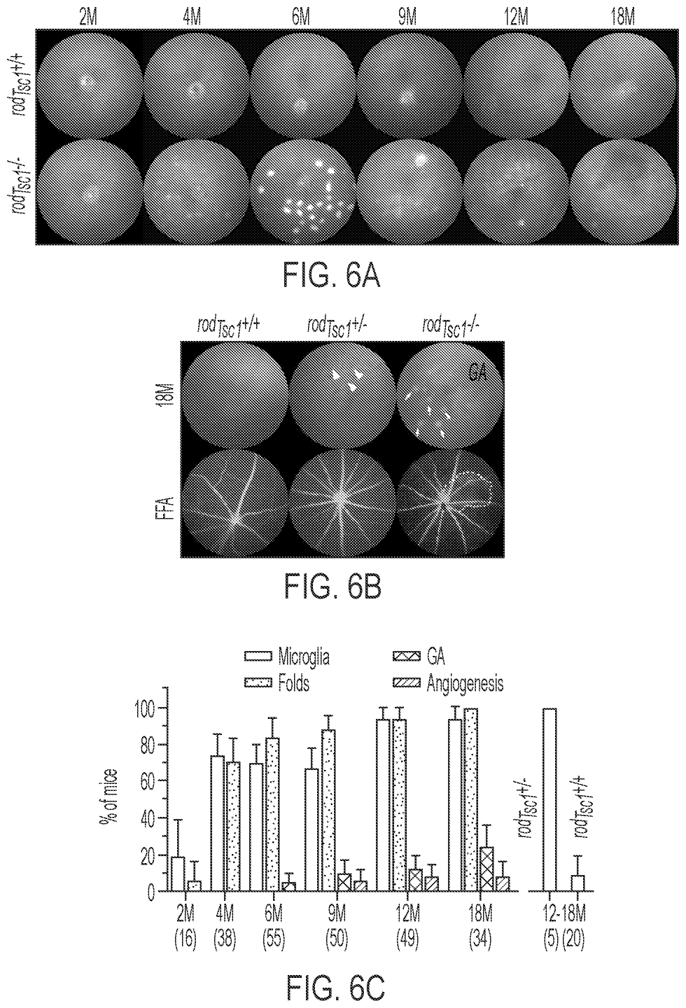

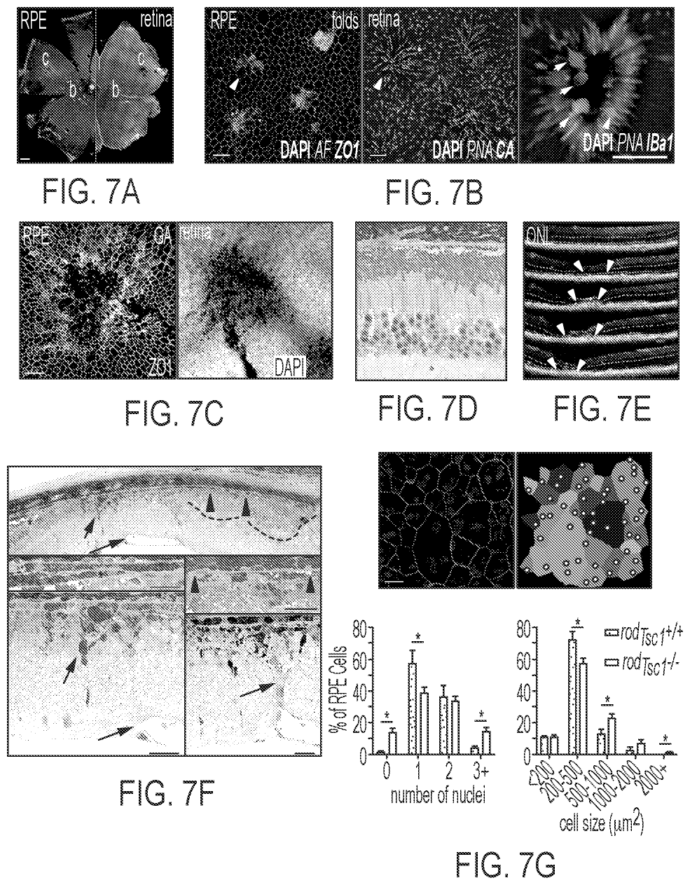

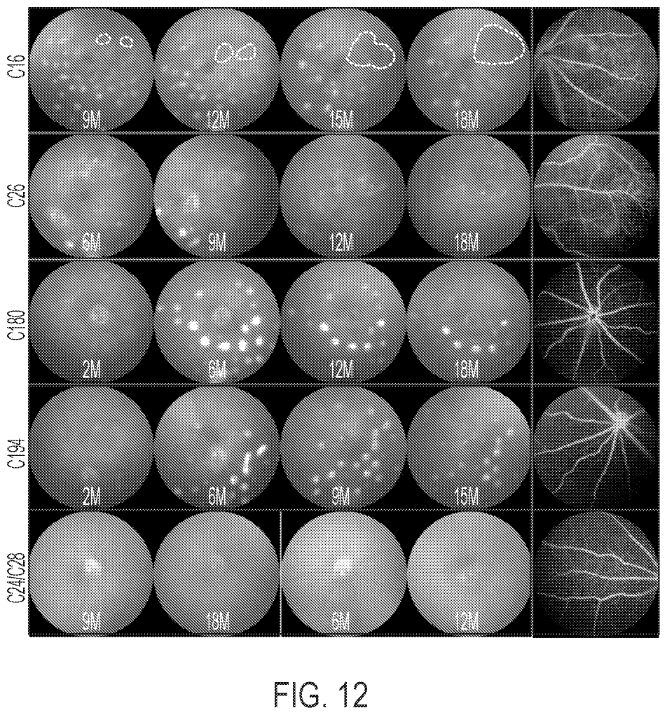

To determine whether ^{rod}Tsc1^{-/-} mice develop advanced AMD-like pathologies, the mice were followed over a period of 18 months (18M) by funduscopy and fluorescein angiography (FIG. 6 and FIG. 12). At 2M, migration and accumulation of microglia into the subretinal space were observed, and at 4M, formation of retinal folds, some of which were filled with microglia were observed (FIG. 13). Flat mount and section analyses revealed highly auto fluorescent RPE cells opposing these folds (FIGS. 7A-7B), which in mice is indicative of acutely compromised or lost RPE cells.

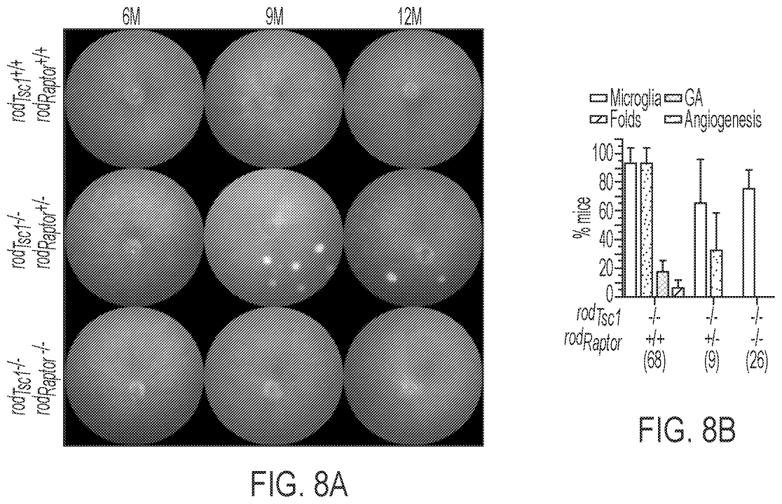

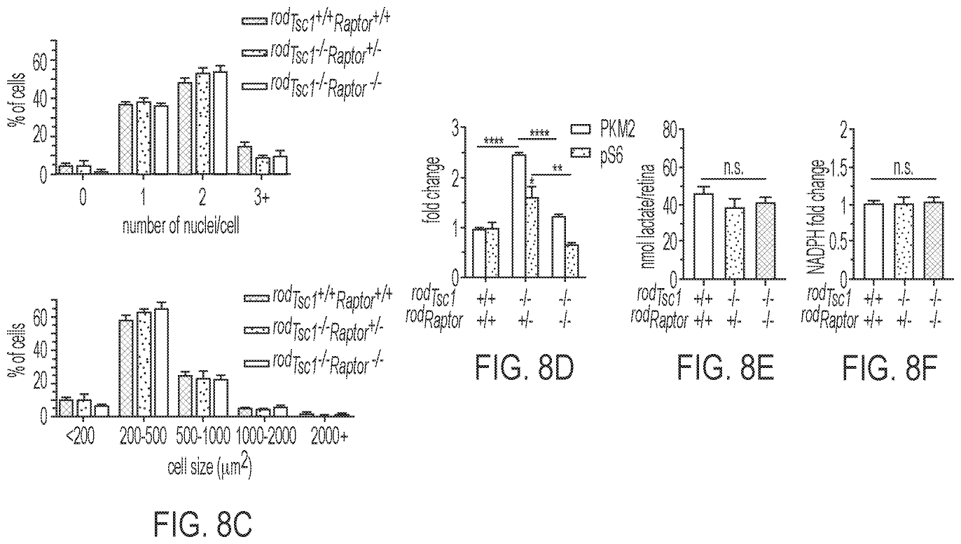

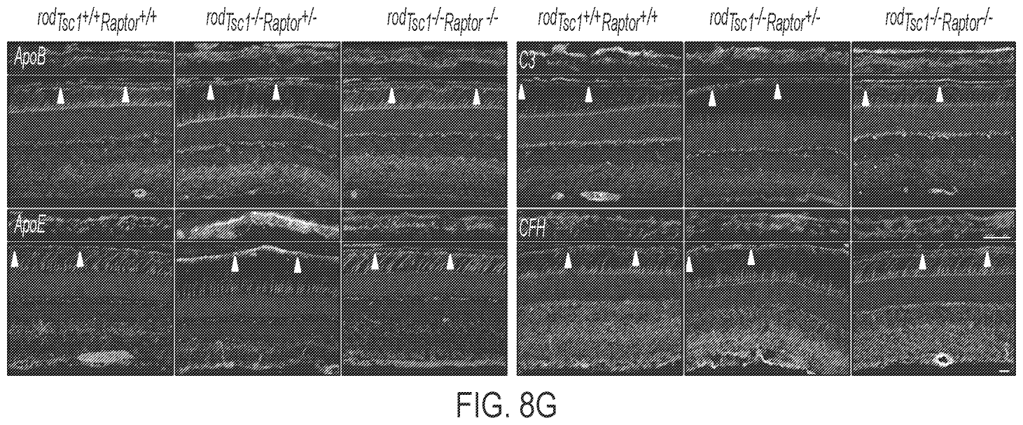

Geographic atrophy was seen in 5% of mice at 6M and 25% of mice at 18M (FIG. 7C). While GA did also overlap with areas of retinal folds, the presence of these folds was not required for GA to develop. Generally, pathologies worsened within the same animal with age (FIG. 12). To confirm that areas of GA correlate with regional PR atrophy and that RPE atrophy precedes PR atrophy, the RPE and corresponding retina were compared by flat mount analyses (FIGS. 8A-8C), identified intermediate RPE pathologies (FIG. 8D) and performed semithin sectioning through regions of GA that were identified by optical coherence tomography (OCT) (FIGS. 8E-8F).

Neovascular pathologies reaching a frequency of 7% by 18M were seen less frequently than GA (FIG. 7C) although most coincided with regions of GA. Retinal neovascular pathologies were regularly detected on semithin sections (FIG. 8F), choroidal neovascular pathologies were not evident on RPE flat mounts. Except for the accumulation of subretinal microglia, none of the heterozygous ^{rod}Tsc1^{+/-} mice nor any of the Cre⁻ littermate control mice (^{rod}Tsc1^{+/+}) developed advanced pathologies (FIGS. 7B-7C). Consistent with this, activation of mTORC1 and the increase in PKM2 expression levels were both minimal in ^{rod}Tsc1^{+/-} mice (FIG. 5C).

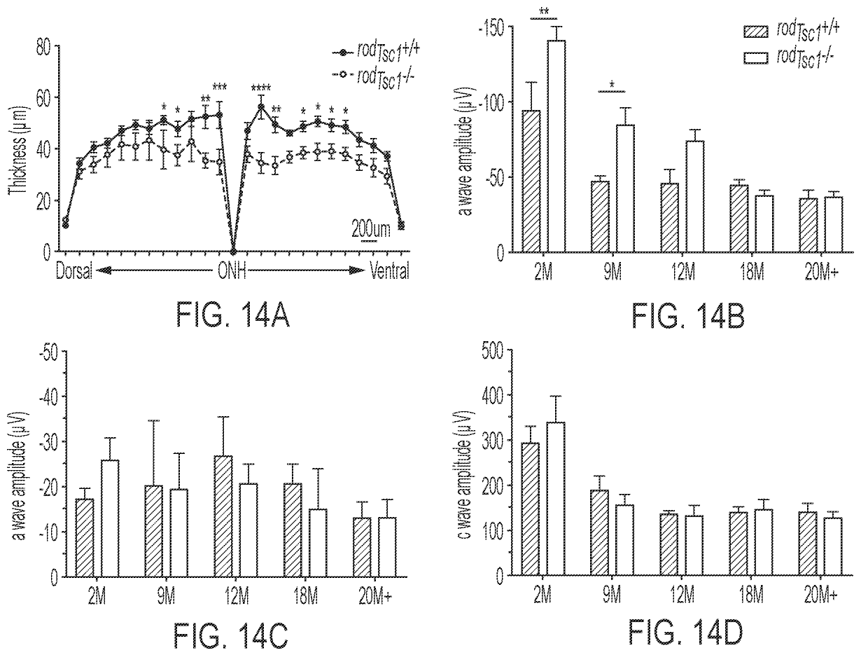

To determine if RPE stress and atrophy also occurred outside regions of GA, the percentage of polynucleated RPE cells was determined and changes in RPE cell size was measured in non-GA areas. At 18M, we found a significant increase in polynucleated and enucleated as well as hypertrophic RPE cells (FIG. 8G). Data indicate that loss of Tsc1 in rods contributes to a widespread RPE pathology that precipitates to regional GA in some animals. It was then investigated whether overall PR survival and function was perturbed. Consistent with a widespread RPE pathology small decrease in the thickness of the PR layer were observed at 18M (FIG. 14A). Rod a-wave amplitudes were higher in ^{rod}Tsc1^{-/-} mice at early time points but declined to the littermate control amplitudes by 18M (FIG. 14B). The early higher amplitude is in line with observations that loss of HK2 leads to a reduction of the scotopic response and a reduction in retinal lactate and NADPH levels. Thus, the early higher amplitude may reflect higher energy availability. Alternatively, increased transcription or translation of phototransduction genes due to increased PKM2 expression or increased mTORC1 activity, respectively, could also account for higher a-wave amplitudes in ^{rod}Tsc1^{-/-} mice. C-wave amplitudes, which reflect in part RPE health, did not differ between ^{rod}Tsc1^{-/-} mice and controls (FIG. 14D). Overall, the data indicates that loss of Tsc1 in rods leads to a slow progressive disease except for areas where advanced pathologies precipitate.

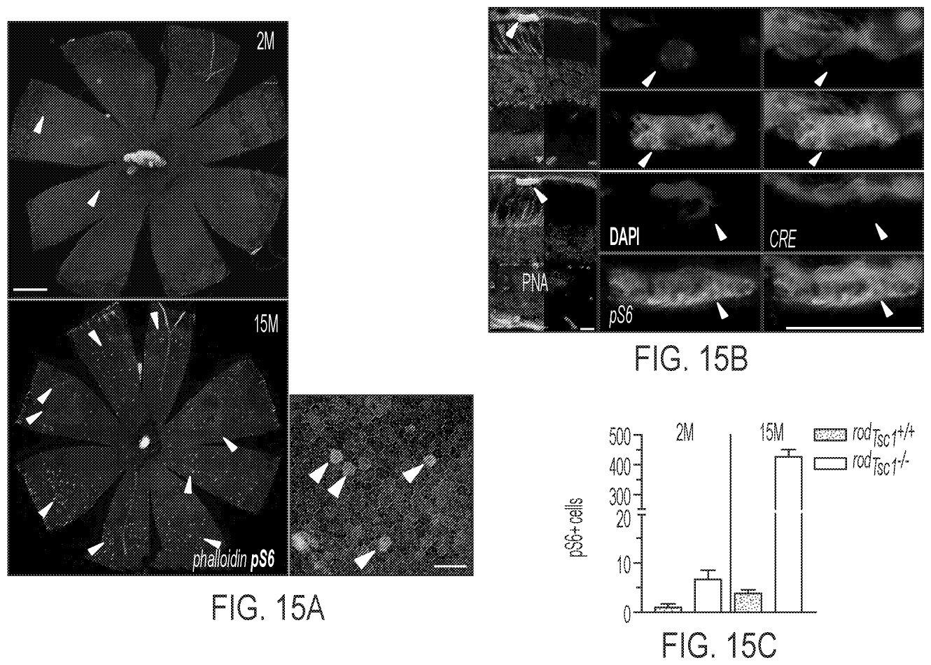

To confirm that GA was not caused by aberrant CRE recombinase expression in the RPE, RPE flat mounts were stained for p-S6. While occasional p-S6 positive cells were seen in both ^{rod}Tsc1^{-/-} mice and controls at 2M (FIG. 15A), CRE recombinase expression was not observed in p-S6 positive cells (FIG. 15B). Additionally, the number of p-S6 positive cells increased dramatically with age (FIG. 15A and 15C). This increase likely reflects an increase in the number of sick RPE cells in ^{rod}Tsc1^{-/-} mice as increased mTORC1 activity in the RPE has been associated with RPE dysfunction, senescence and cell loss.

^{rod}Tsc1^{-/-} Mice also Display Early Disease Features

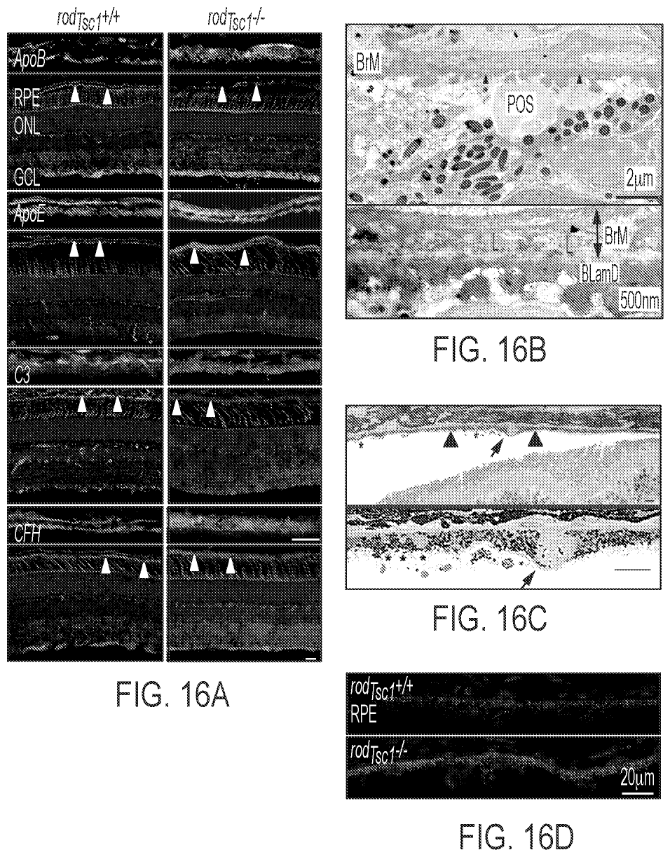

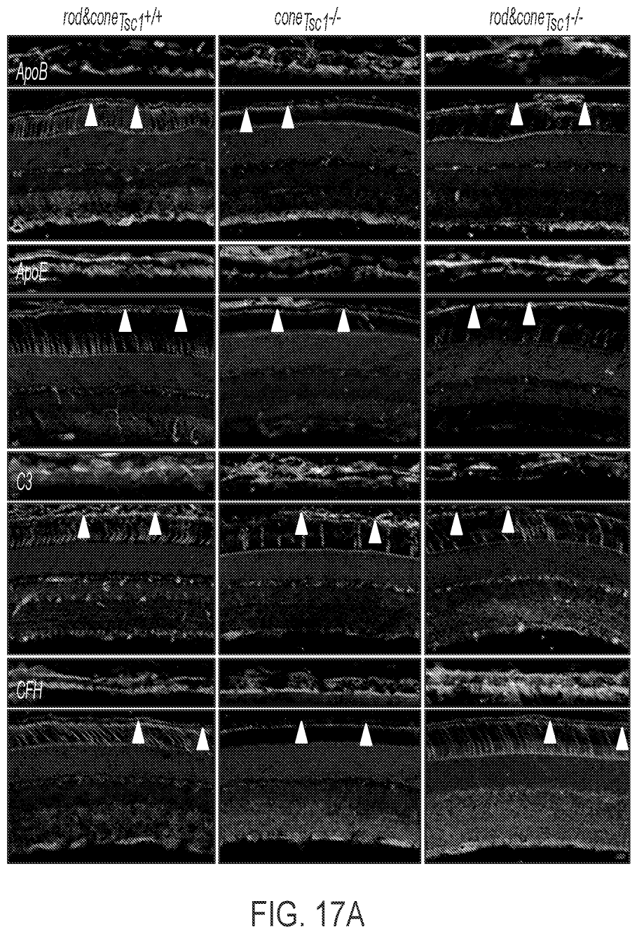

The metabolic demands of PRs have been proposed to contribute to lipoprotein accumulation and drusen formation. To determine if the metabolic changes induced in PRs also contributes to lipoprotein accumulation, distribution of ApoB and ApoE at the BrM was investigated. Accumulation of both lipoproteins at the RPE basal lamina and BrM was observed, independent of any advanced pathology (FIG. 16A). Electronmicroscopy (EM) analyses revealed neutral lipids within the BrM, as well as basal laminar deposits and thickened BrM in areas of GA (FIG. 16B). However, drusen-like deposits were not seen, rather, basal mounds were quite common (FIG. 16C). Increased autofluorescence was observed in the RPE of ^{rod}Tsc1^{-/-} mice, indicative of increased lipofuscin accumulation (FIG. 16D).

A uniform downregulation of C3 was observed at the BrM, and a uniform upregulation of CFH in ^{rod}Tsc1^{-/-} mice (FIG. 16A). Data indicate that these early disease features, which are induced by activation of mTORC1 in rods, occur uniformly across the tissue independent of the presence of any advanced pathology.

AMD-Like Pathologies are Dependent on the Dose of Activated mTORC1

To test the requirement of mTORC1 to the pathologies seen, mice with simultaneous deletion of Tsc1 and the mTORC1 adaptor protein Raptor (referred to ^{rod}Tsc1^{-/-rod} Raptor^{-/-} mice) were obtained. Fundus imaging reveled no pathology except for the accumulation of microglia in 76% of mice aged between 12-18M (FIGS. 8A and 8B). Even heterozygous Raptor mice (^{rod}Tsc1^{-/-} Raptor^{-/+}) did not develop any GA or neovascular pathologies by 12M (FIG. 8B). However, retinal folds were present albeit at lower frequency. The absence of any severe pathology was in line with the quantification of polynucleated RPE cells and RPE cell size, which revealed no substantial difference among these lines at 12M (FIG. 8C). Western blot analyses for p-S6 and PKM2 confirmed the reduction in mTORC1 activity (FIG. 8E). While p-S6 levels in ^{rod}Tsc1^{-/-rod} Raptor^{+/-} showed a dose dependent decline when compared to in ^{rod}Tsc1^{-/-} mice, PKM2 levels remained similar to PKM2 levels in ^{rod}Tsc1^{-/-} (compare FIG. 8D with FIG. 5C). In contrast, lactate and NADPH levels remained at the levels of Cre⁻ controls in heterozygous ^{rod}Tsc1^{-/-rod} Raptor^{+/-} mice (FIGS. 8E and 8F). To determine to which extend this affected the early pathologies the accumulation of ApoB, ApoE, C3 and CFH was analyzed. While accumulation of these markers was restored to normal in ^{rod}Tsc1^{-/-rod} Raptor^{-/-} mice, heterozygous ^{rod}Tsc1^{-/-rod} Raptor^{+/-} mice displayed a more intermediate phenotype (FIG. 8G). ApoB showed almost no accumulation, while ApoE accumulation was similar to that seen in ^{rod}Tsc1^{-/-} mice. Similarly, CFH showed very little accumulation and C3 was substantially reduced. The data indicate that the development of early and late pathologies is driven in a dose-dependent manner by increased mTORC1 activity.

RPE Phagocytosis is Perturbed in ^{rod}Tsc1^{-/-} Mice

Impaired RPE lysosomal activity has been associated with AMD. The uniform nature of

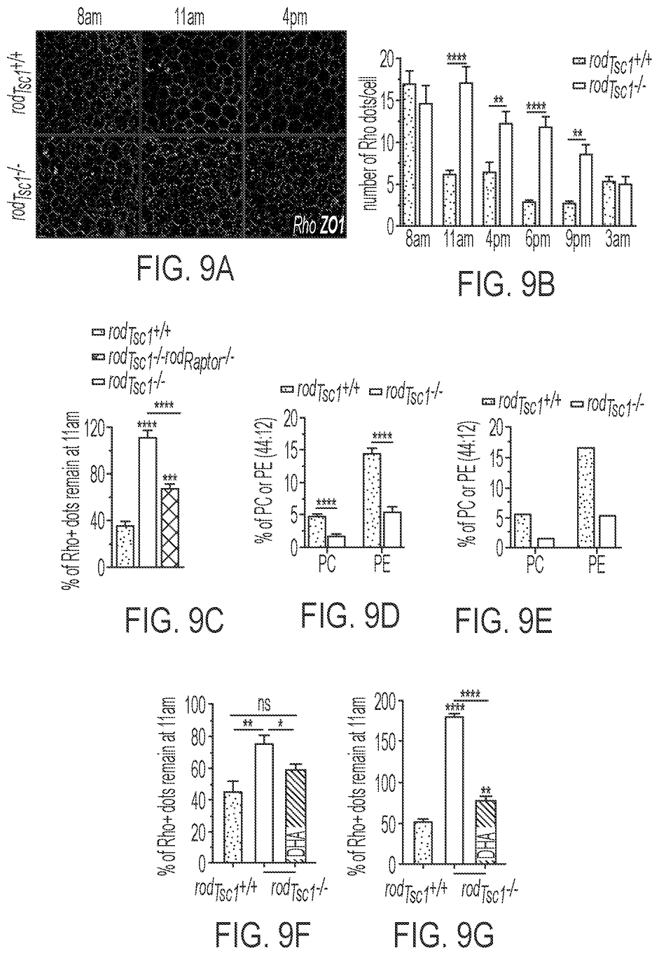

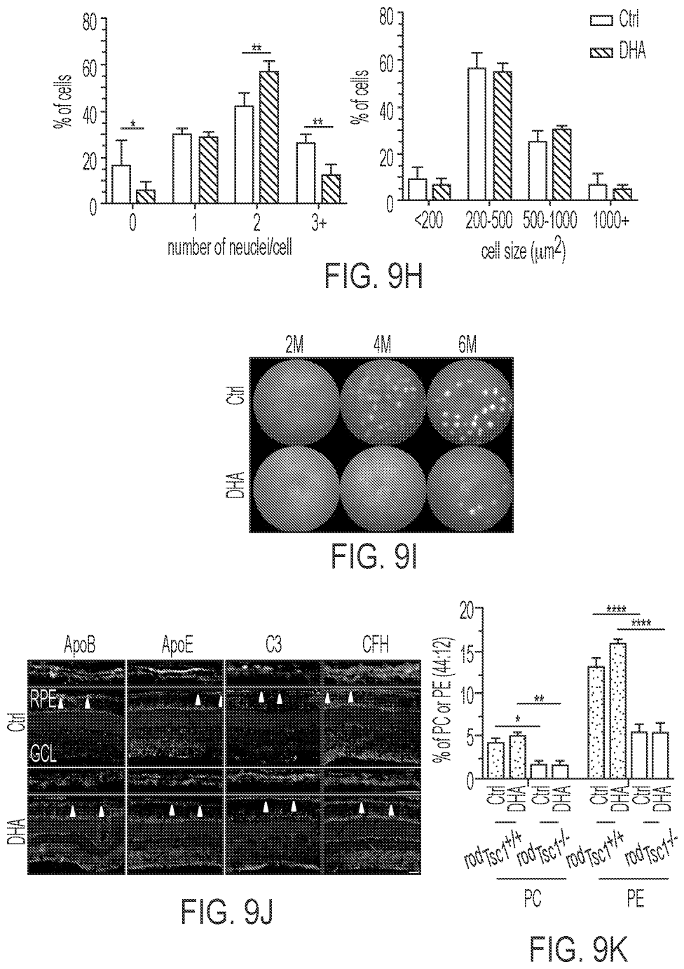

RPE cell stress led us to investigate if POS clearance was perturbed in the ^{rod}Tsc1^{-/-} mice. Since shedding of rod POSs is circadian, clearance can be monitored over time on RPE flat mounts stained for the rhodopsin protein. Rod POS clearance was observed to be significantly slowed at 2M in ^{rod}Tsc1^{-/-} mice and was rescued in ^{rod}Tsc1^{-/-rod} Raptor^{-/-} mice, indicating that the effect was due to increased mTORC1 activity in rods (FIGS. 9A-9C)

POSs are rich in lipids and mTORC1 is known to regulate lipid synthesis. To determine a cause for the delayed POS clearance by the RPE the retinal lipid composition of ^{rod}Tsc1^{-/-} mice was profiled. A ˜3-fold decrease was observed in di-DHA (44:12) containing phosphatidylethanolamine (PE) and phosphatidylcholine (PC) lipids in total retinal (FIG. 9D) and POS preparations (FIG. 9E). To test if this drop in di-DHA PE and PC lipids contributes to the delay in POS clearance ^{rod}Tsc1^{-/-} mice were fed a diet enriched with 2% DHA. Feeding ^{rod}Tsc1^{-/-} mice a 2% DHA enriched diet from weaning onwards improved POS clearance at 2M (FIG. 9F). To test if delayed POS clearance can also be improved once the delay has occurred, 6M old ^{rod}Tsc1^{-/-} mice were fed the DHA enriched diet for 2 weeks. This had an even more pronounced effect, as POS clearance was more affected at 6M (FIG. 9G).

To determine if dietary DHA also affected overall RPE health, mice were kept on the DHA diet from weaning onwards until 6M. This reduced the percentage of polynucleated RPE cells (FIG. 9H), improved fundus pathologies (FIG. 9I), prevented the accumulation of ApoB, ApoE and CFH, and restored C3 expression (FIG. 9J). Differences in RPE hypertrophy were not evident, likely because in younger mice hypertrophy is not as pronounced yet. None of 12 DHA-fed mice (n=12) developed any GA by 6M, while 1 out of 6 mice on the control diet did. Re-profiling of the retinal lipids after 10 weeks of DHA feeding revealed that levels of di-DHA containing PE and PC lipids were not restored. This indicates that DHA must have acted directly on the RPE to improve overall PRE health (FIG. 9K). In all, the data indicate that activated mTORC1 in rods affects the retinal lipid composition, which affects overall RPE health.

Cones Contribute Differently than Rods to Disease

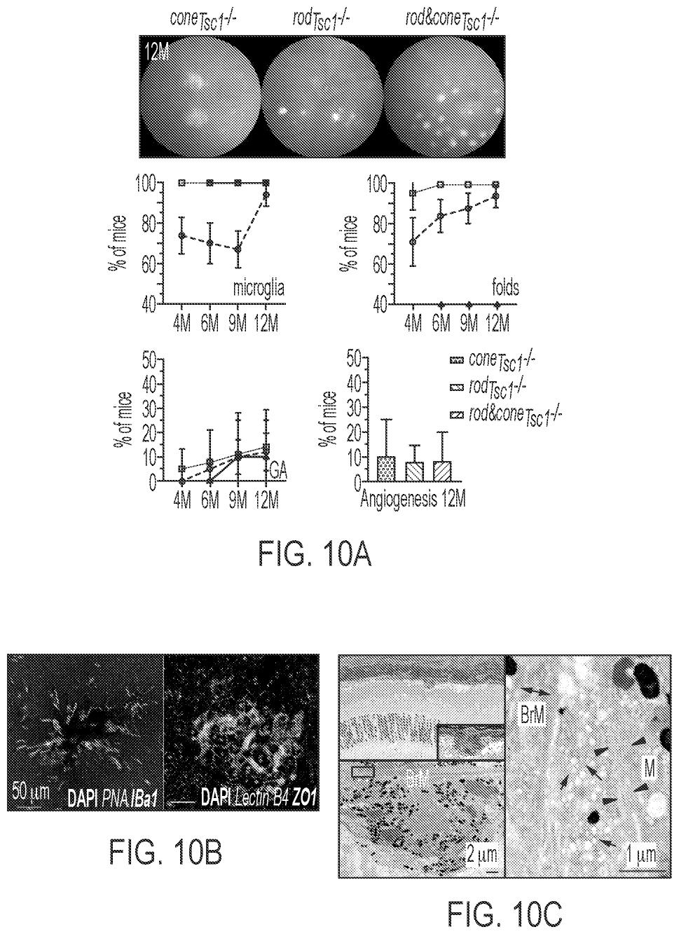

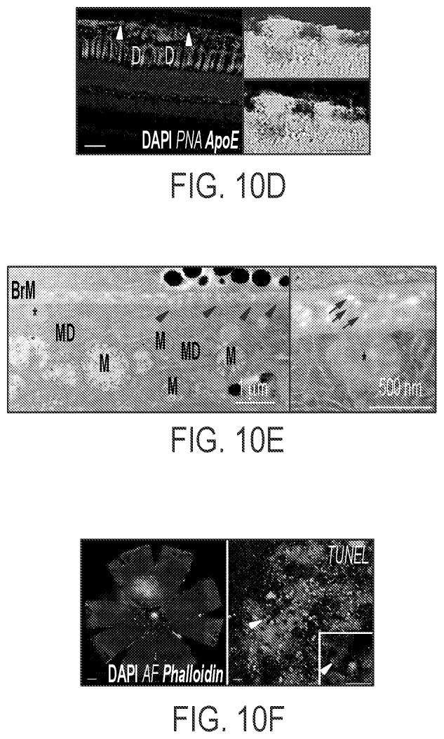

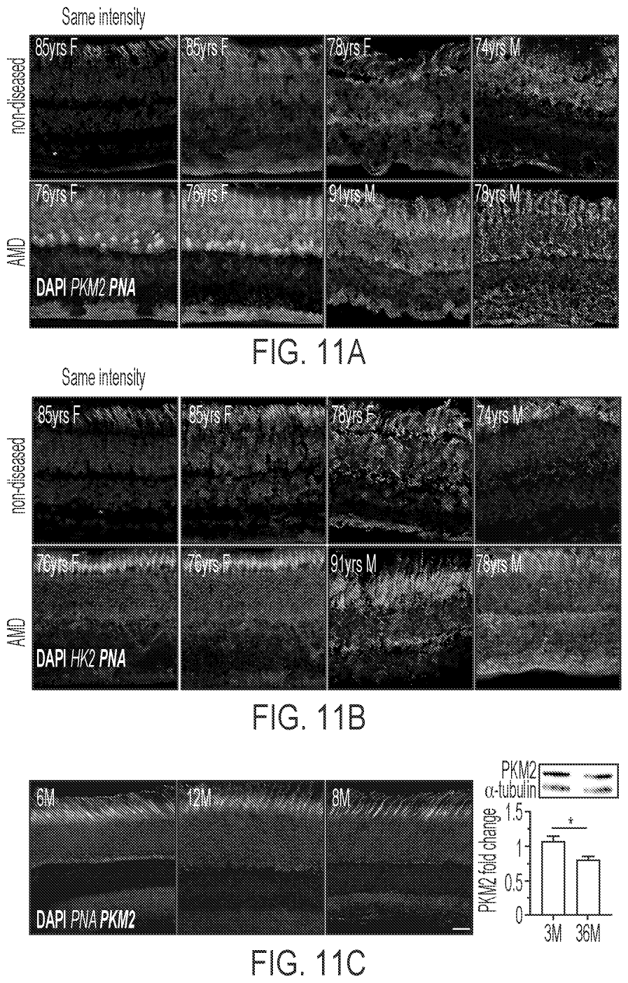

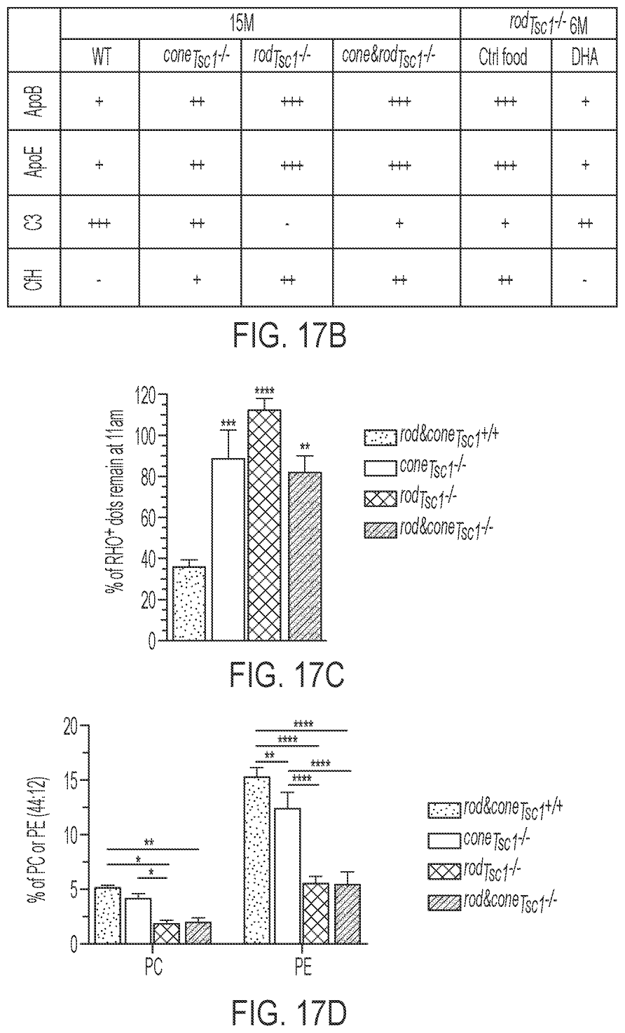

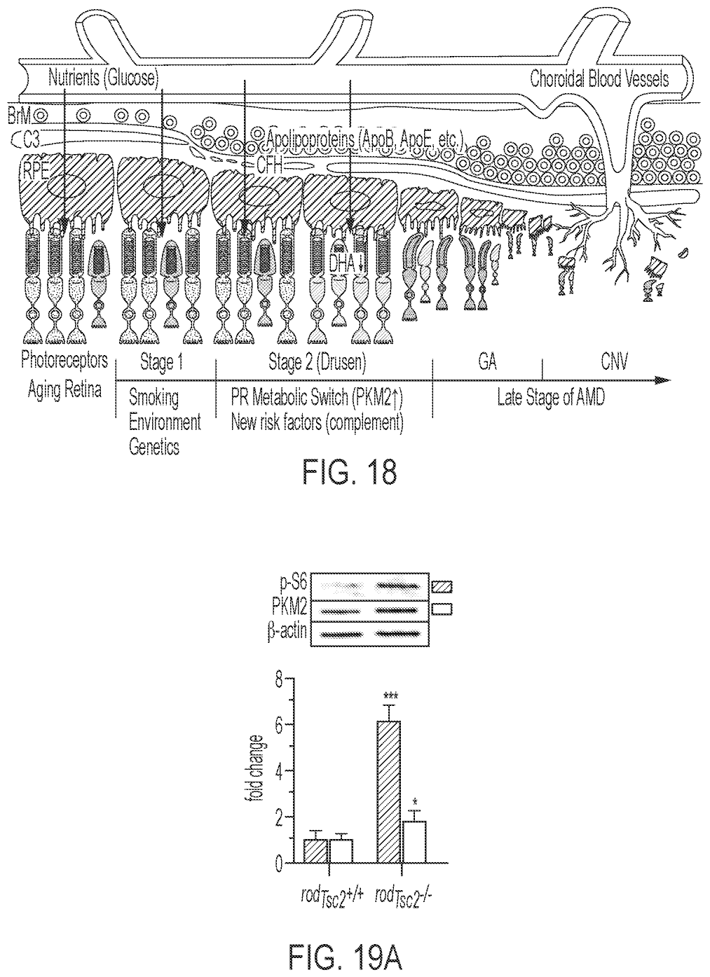

A cell line with a cone-specific deletion of Tsc1 (^{cone}Tsc1^{-/-}) and one with a rod-&-cone deletion (^{cone&rod}Tsc1^{-/-}) were obtained. Funduscopy and angiography revealed that ^{cone}Tsc1^{-/-} mice develop similar pathologies without the formation of retinal folds (FIG. 10A). Combining the metabolic changes in rods and cones did not increase the overall frequency of advanced pathologies by 12M. However, advanced pathologies started to occur already at 4M (FIG. 10A). Choroidal neovascular pathologies in ^{cone}Tsc1^{-/-} mice were easier to identify on RPE flat mounts when compared to ^{rod}Tsc1^{-/-} mice (FIG. 10B). ^{cone}Tsc1^{-/-} and ^{cone&rod}Tsc1^{-/-} mice also developed large drusen-like deposits that were positive for ApoE (FIGS. 10C and 10D). Such large deposits were not seen in ^{rod}Tsc1^{-/-} mice. EM analyses revealed that loss of Tsc1 in cones was sufficient to cause accumulation of small lipoprotein vesicles, reminiscent of basal linear deposits, within the BrM (FIG. 10E), which may explain the difference in deposit size. Finally, areas of GA were generally larger in ^{cone&rod}Tsc1^{-/-} mice when compared ^{rod}Tsc1^{-/-} or ^{cone}Tsc1^{-/-} mice (FIG. 10F). This allowed visualization of ongoing RPE atrophy by TUNEL (FIG. 10F). All other pathologies such as uniform accumulation of lipoproteins and changes in C3 and CFH expression were similar between all three lines, with the ^{cone}Tsc1^{-/-} mice displaying the least pronounced changes (FIGS. 17A-17B). Rod POS clearance was also affected in ^{cone}Tsc1^{-/-} mice and loss of Tsc1 in cones affects rod POS clearance (FIG. 17C). Di-DHA PE lipids were also significantly reduced in ^{cone}Tsc1^{-/-} mice (FIG. 17D), indicating that any reduction in di-DHA PE lipids may affect RPE health. Together, the data indicate that there are distinct mechanisms between rods and cones that contributed to advanced AMD pathologies, which is in line with observations in humans.

### Example 3

Age-related Macular Degeneration (AMD) is one of the leading causes for visual impairment in the elderly. The disease is multi-factorial including genetic and non-genetic risk factors. Omega-3 fatty acid rich foods, in particular Docosahexaenoic acid (DHA) rich foods, have been found to reduce disease risk (e.g., Souied, E. H. et al. Omega-3 Fatty Acids and Age-Related Macular Degeneration. Ophthalmic Res 55, 62-69, (2015)). Similarly, high DHA plasma levels correlate with reduced disease risk (e.g., Merle, B. M. et al. High concentrations of plasma n3 fatty acids are associated with decreased risk for late age-related macular degeneration. J Nutr 143, 505-511, (2013)). Moreover, individuals with AMD have a 30% reduction in retinal DHA levels. Despite these findings and the identification of over 30 risk alleles. no animal model generated to date has faithfully recapitulated the complex disease progression of AMD11, nor is the role of DHA in disease pathogenesis fully understood.

AMD is considered a retinal-pigmented epithelium disease (RPE). During the early disease stages deposits, referred to as drusen, form between the RPE and the underlying basement membrane, known as the Bruch's membrane (BrM). Over time these deposits grow in number and size affecting RPE health. Eventually, affected individuals' progress to one of two advanced forms of the disease, namely geographic atrophy (GA) or choroidal neovascularization (CNV). In GA, large areas of confluent RPE loss leads to secondary photoreceptor (PR) death as the RPE is involved in transferring nutrients from the adjacent choroidal vasculature to PRs. In CNV, the choroidal vasculature breaks through the Bruch's membrane and the RPE resulting in retinal edemas that cause PR loss. While CNV can be treated with vascular endothelial growth factor (VEGF) inhibitors to prevent excessive edema formation, there is no treatment for GA or to prevent progression from the earlier drusen stage to the advanced stages. The reason for this is a lack of understanding as to the cause and progression for the disease. Since 85% of advanced AMD patients suffer from GA, there is an unmet need to develop treatments that either prevent disease progression from the drusen stage to the advanced stages or further progression of GA.

Photoreceptors have long been considered a bystander of the disease pathogenesis, even though PR metabolism has been linked to both, the early and the late disease stage. Studies on the distribution of the lipoprotein rich drusen deposits, which are a marker of the early disease stage, revealed that the location of the two major types of pathological drusen seen in AMD patients mirrors the density distribution of cone and rod PRs. Macular translocation procedures, which were used to treat the late-disease stage of GA, indicate that PRs can also cause this condition. Patients whose retina were rotated to move macular cones away from an area of dying RPE to an area of healthy RPE redeveloped GA where the cones were translocated. In both cases the high and different metabolic demands of cones and rods have been proposed to underlie the formation of these pathologies. Therefore, whether the metabolic demands of PRs differ in patients with AMD was investigated. Increased expression of two key metabolic PR genes was found, suggesting that PRs are adapting to a nutrient shortage. To determine the long-term effects of such metabolic adaptation, mammalian target of rapamycin complex 1 (mTORC1)16 in mouse PRs was constitutively activated, since mTORC1 regulates cell metabolism under nutrient stress. This was achieved by deletion of the tuberous sclerosis complex 1 protein (TSC1). It was found that the onset of pathologies are age and mTORC1 dependent, which is reminiscent of those seen in humans, including drusen, GA and CNV. The mouse model described in this disclosure is thus the first animal model that develops all the cardinal features of the early as well as the late disease stages. Importantly, disease progression in our mouse model is dependent on dietary DHA levels and, similarly to humans, our mice display a reduction in specific di-DHA containing retinal phospholipids. Our mice thus offer us the opportunity to identify new disease-causing mechanisms downstream of mTORC1 that contribute to disease progression as well as test the efficacy of potential therapeutic candidates in delaying disease progression.

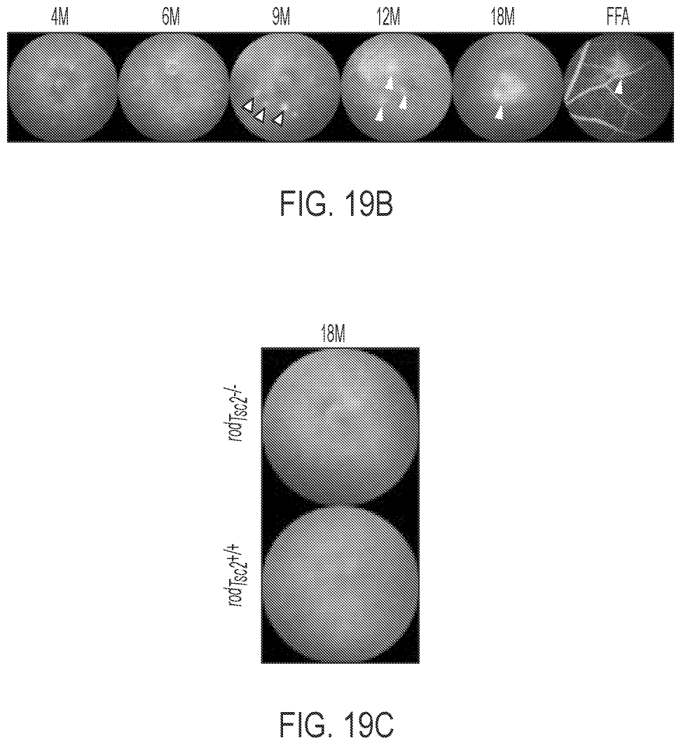

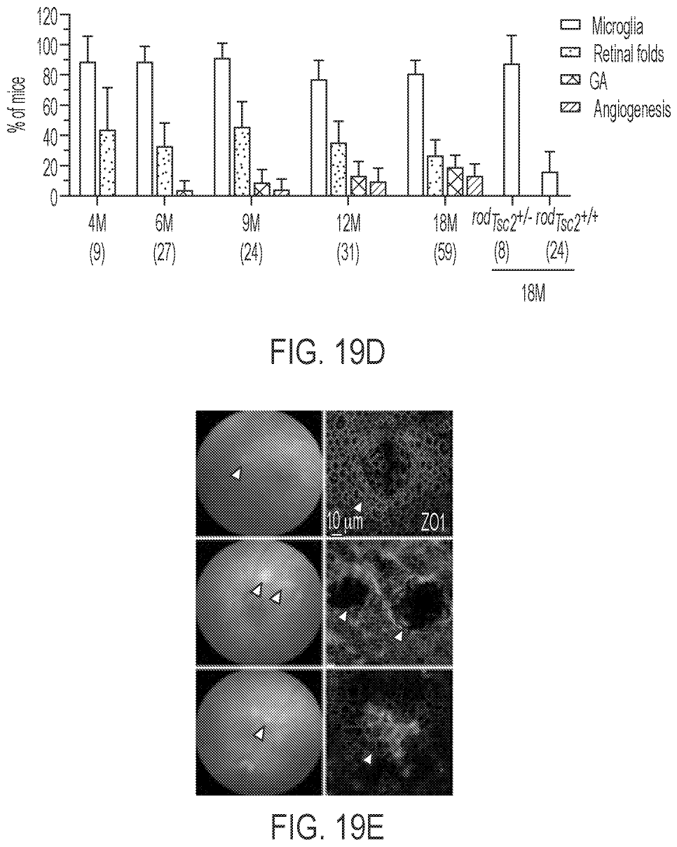

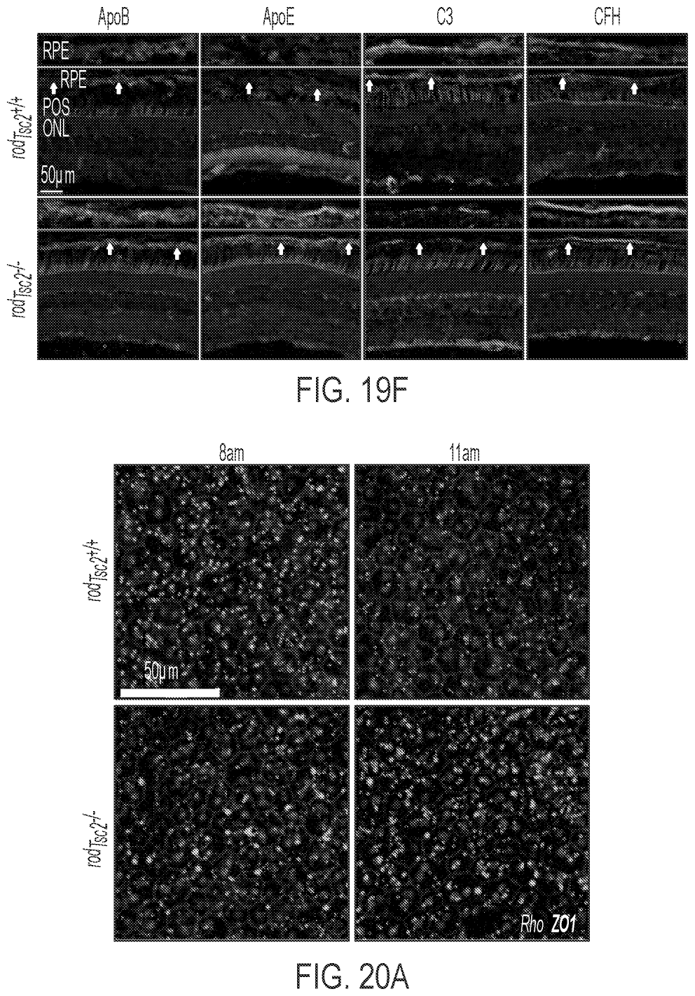

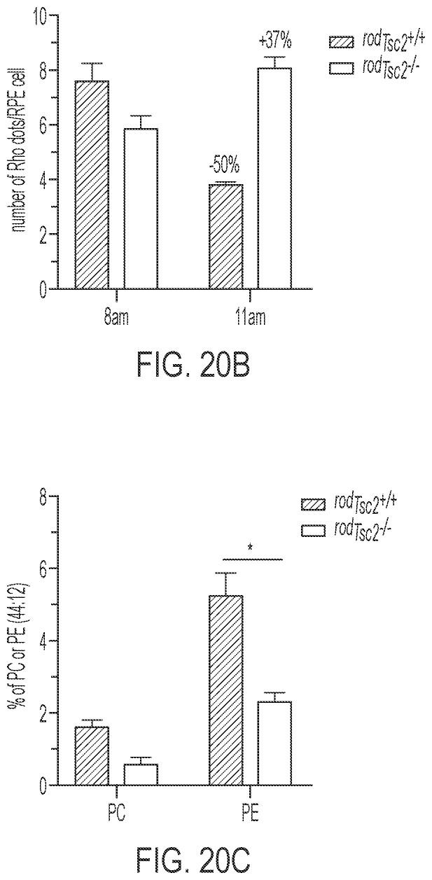

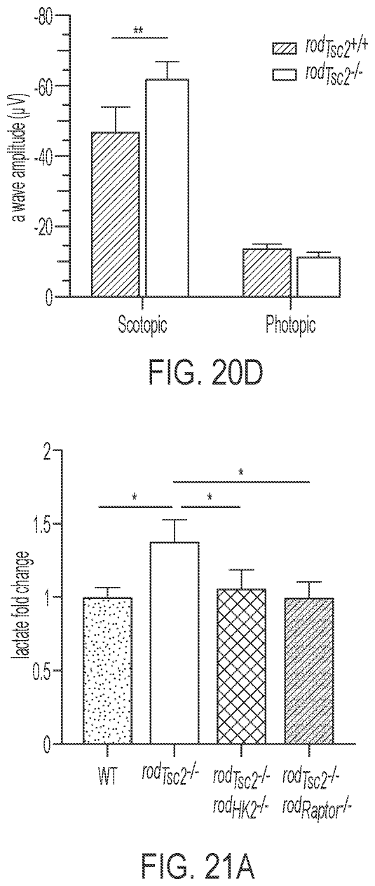

To mimic the adaptive changes suggestive of a nutrient deprivation seen in PRs of AMD patients, mTORC1 was constitutively in the mice, since mTORC1 regulates cell metabolism under nutrient stress. The metabolic processes regulated by mTORC1 include glycolysis, fatty acid synthesis, protein translation, autophagy and the activity of the second mTOR complex, mTORC2, which also regulates AKT activity. It was previously confirmed that mTORC1 activity is required for the pathologies seen upon loss of TSC1 in rods. Additionally, to confirm that the pathologies were not associated with unknown functions of the TSC1 protein, TSC complex was disrupted by selectively removing the second TSC complex protein, namely TSC2, in rods (^{rod}Tsc2^{-/-}). This resulted in the same overall pathologies and disease progression as loss of TSC1 in rods (FIGS. 19A-10F). These mice also showed a delay in photoreceptor outer segment (POS) digestion, a reduction in di-DHA containing phosphatidylethanolamine (PE) and phosphatidylcholine (PC) lipids and an increase in the scotopic electroretinogram (ERG) recordings (FIGS. 20A-20D). Increase in mTORC1 activity and in aerobic glycolysis were confirmed by western blotting for phosphorylated ribosomal protein S6 (p-S6) and pyruvate kinase muscle isozyme M2 (PKM2), both of which showed higher levels in (^{rod}Tsc2^{-/-}) mice (FIG. 19A).

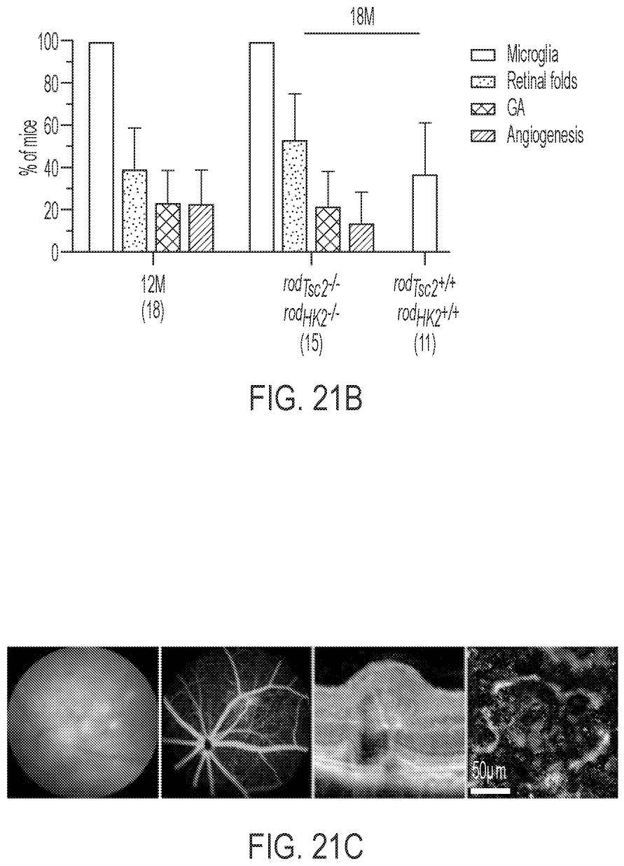

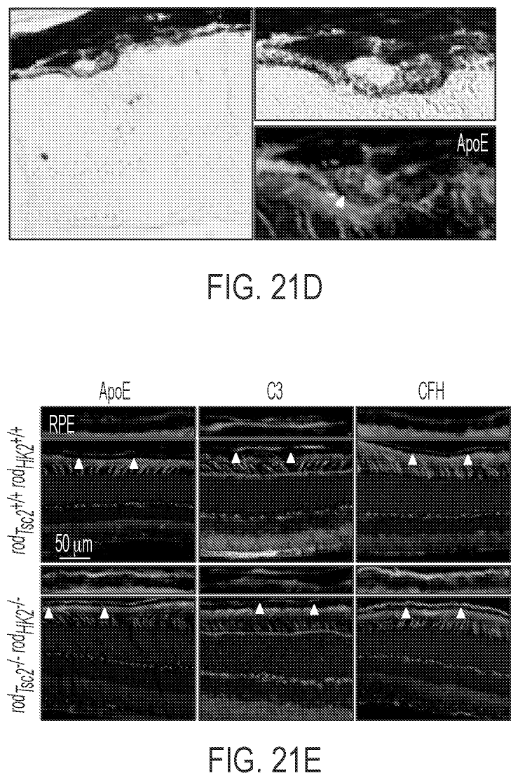

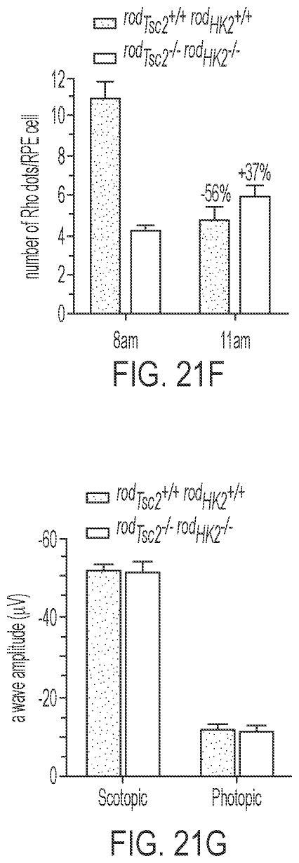

Next, to determine which aspect downstream of mTORC1 is required for early and late-stage pathologies to develop, the contribution of glycolysis was tested by abolishing in the context of TSC complex disruption, also the activity of Hexokinase-2 (HK2) (^{rod}Tsc2^{-/-rod} HK2^{-/-}). This reduced the increase in lactate levels caused by disruption of the TSC complex (FIG. 21A), and the levels of the scotopic ERG response (FIG. 21G) thereby reversing some of the alterations in glycolysis induced by activation of mTORC1. However, as the data show, loss of HK2 in the context of hyperactivated mTORC1, still leads to the same pathologies as seen with loss of TSC1 in rods or loss of TSC2 (FIGS. 19A-20D) in rods (FIGS. 21A-21F).

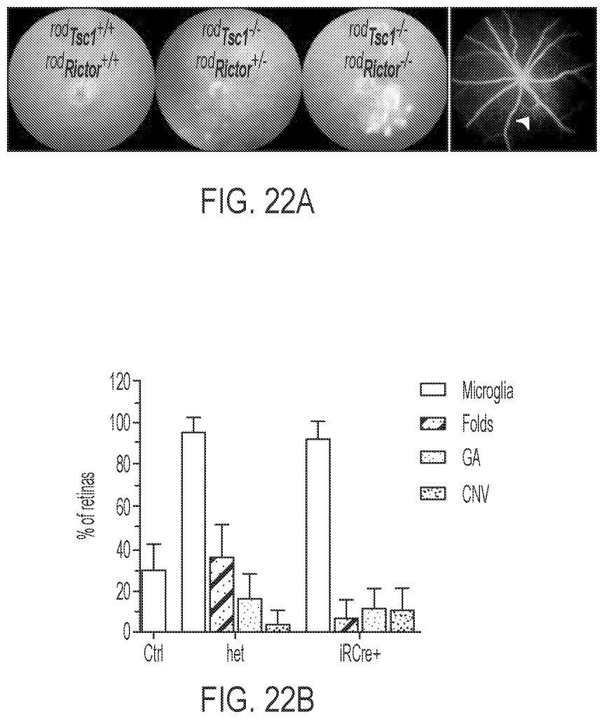

Similarly, to test the contribution of the mTORC2 complex and AKT together, mice with simultaneous deletion of TSC1 and the mTORC2 adaptor protein Rictor (^{rod}Tsc1^{-/-rod} Rictor^{-/-}) were generated. Similar to ^{rod}Tsc2^{-/-rod} HK2^{-/-} mice, ^{rod}Tsc1^{-/-rod} Rictor^{-/-} mice still develop advanced AMD pathologies (FIGS. 22A-22B), indicating that changes in glycolysis, AKT signaling or mTORC2 activity are not what contributes to advanced AMD.

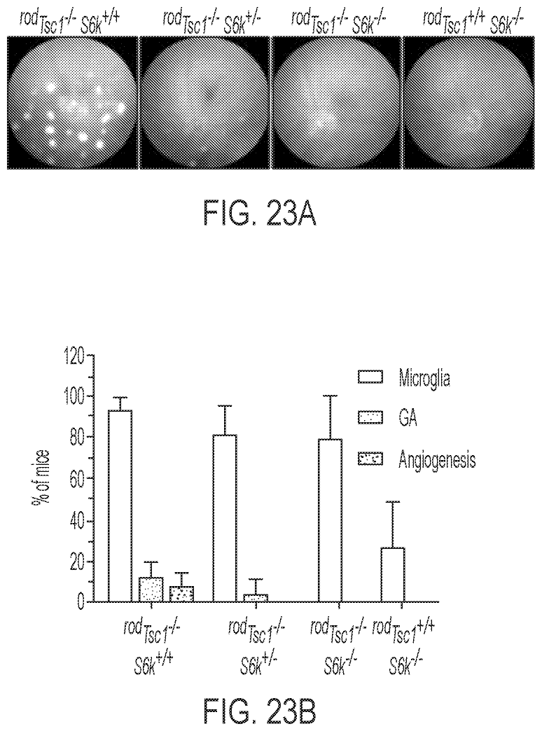

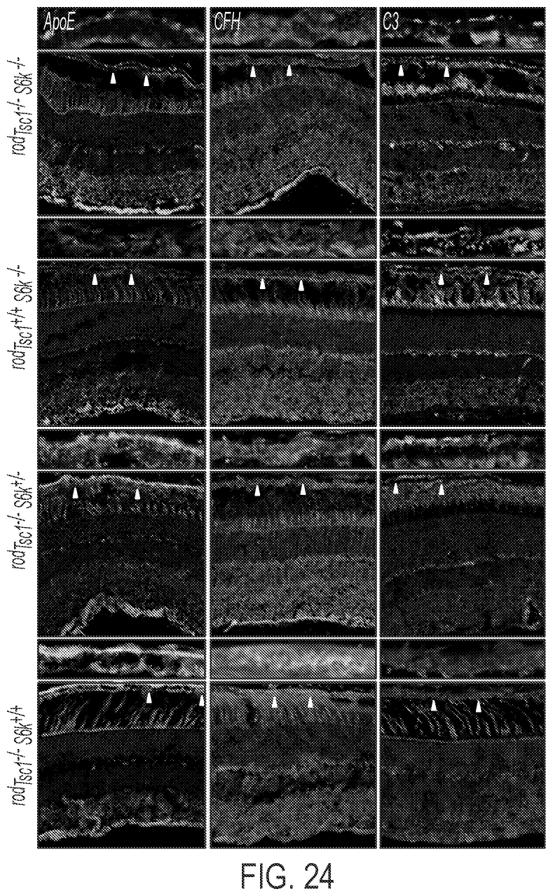

The remaining processes regulated by mTORC1 are lipid synthesis, protein synthesis and autophagy. Because autophagy and overall increased protein synthesis are directly regulated by mTORC1, while most of the lipid synthesis pathways are regulated by mTORC1 in an S6K1-dependant manner. To test this theory, mice with loss of TSC1 and S6K1 (^{rod}Tsc1^{-/-} S6K1^{-/-}) were generated. It was found that removal of S6K1 in the context of TSC1 loss completely inhibits the development of any pathologies (FIGS. 23A-23B). Consistent with this, markers of the early disease stages such as accumulation of Apolipoprotein E (ApoE), as well as complement factor H (CFH), or reduction in complement factor 3 (C3) expression were all restored to their corresponding age-matched wild-type levels at the Bruch's membrane (BrM) and RPE interphase in eyes of ^{rod}Tsc1^{-/-} S6K1^{-/-} mice (FIG. 24). To determine if there is a dose dependent effect, heterozygous ^{rod}Tsc1^{-/-} S6K1^{+/-} mice were also tested. It was found that loss of one allele of S6K1 still prevented neovascular pathologies to occur and dramatically reduced the frequency of GA (FIGS. 23A-23B). In agreement with this data, the expression changes seen with the early disease markers was mixed (FIG. 24). ApoE showed the same accumulation as seen in diseased mice with two S6K1 wild-type alleles. In contrast CFH and C3 levels were intermediate with less CHF accumulation and more C3 expression when compared to diseased mice with two S6K1 wild-type alleles (FIGS. 21A-21G). In summary, the data suggest that changes in lipid synthesis and not in autophagy or overall protein synthesis underlie the development and progression of AMD. Importantly, AMD pathologies can be alleviated or prevented in a dose dependent manner by decreasing S6K1 expression. This indicates that any inhibition of S6K1 function or expression is beneficial to delay disease progression. Therefore, complete inhibition of S6K1 function is not required for a successful therapeutic approach.

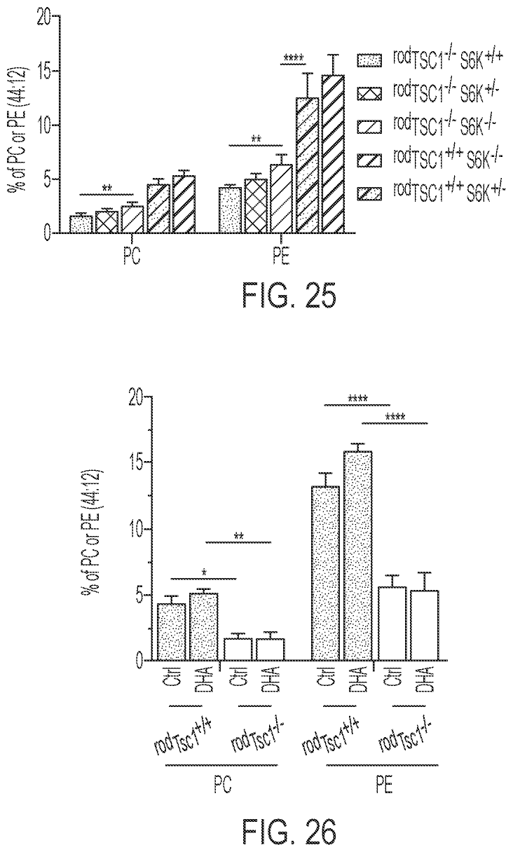

To test if S6K1 loss does indeed affect lipid synthesis, the retinal phospholipids was profiled. In mice with TSC1 loss, a significant reduction in di-DHA containing phosphatidylethanolamine (PE) and phosphatidylcholine (PC) lipids was observed. Similarly, a strong reduction of di-DHA PE and PC lipids was found in mice with loss of TSC2 in rods (FIGS. 20A-20D), although baseline levels are different between the two strains. This likely indicates a difference in the strain background rather than a difference due to loss of TSC1 versus loss of TSC2. Interestingly, loss of S6K1 resulted in a dose dependent increase of these two di-DHA containing phospholipids regardless of the hyperactivation of mTORC1 (FIG. 25). The increase with complete loss of S6K1 was approximately ˜30%. This parallels the decrease in retinal DHA levels found in patients with AMD (˜30%). Since feeding mice a DHA enriched diet prevents disease progression the data suggest that part of the protective effect mediated by S6K1 loss might be due to the increase in retinal DHA levels. The protective effect of dietary DHA is further underscored by over 15 epidemiological studies as well as a study that associated high omega-3 fatty acid levels in the blood to a reduction in disease risk. Finally, a small study in humans that used 5× higher levels of omega-3 fatty acids than the NIH sponsored AREDS2 study showed a protective effect of dietary omega-3 fatty acids like DHA in reducing the risk of disease progression. Importantly, in our mice with loss of TSC1, retinal di-DHA levels of PE and PC lipids did not increase following DHA feeding (FIG. 26) although pathologies were significantly alleviated. DHA may act directly on the RPE to improve overall RPE health. This approach requires high levels of DHA supplementation. In contrast in control wild-type mice, DHA feeding increased retinal di-DHA levels of PE and PC lipids to a similar extend than seen with loss of S6K1 expression. The genetic approach of reducing S6K1 expression levels or its activity allows thus for increasing DHA levels in the retina without the need for excess dietary supplementation. Since the RPE phagocytoses the POSs that are rich in DHA increasing retinal DHA levels by S6K1 reduction or inhibition is more beneficial that increasing DHA levels in the RPE through high dose dietary DHA supplementation. Additionally, since the reduction in retinal di-DHA levels caused by excess S6K1 activity is unlikely to be the sole cause for the development and progression of AMD, reducing S6K1 therapeutically by knockdown or inhibition of its function, is a better therapeutic approach.

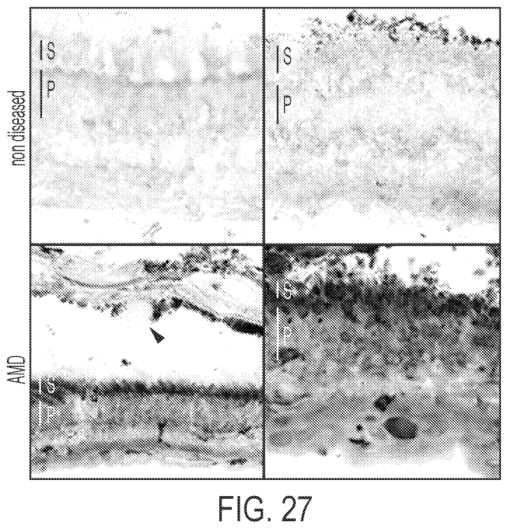

Finally, to verify that S6K1 activity is indeed increased in patients with AMD, an immunohistochemistry analyses for p-S6 on retinal sections of non-diseased individuals and patients with AMD was performed. p-S6 is a bona-fide readout of S6K1 activity as it is one of the canonical targets of S6K1. Similarly, S6K1 is a bona fide target of mTORC1. Therefore, increased levels of p-S6 means that there is increased mTORC1 and increased S6K1 activity. The results showed significantly increased levels of p-S6 in PRs of AMD patients (FIG. 27) indicating that the proposed mechanism of action is indeed correct. Increased activation of mTORC1 in PRs of AMD patients contributes to advanced pathologies through increased activation of S6K1, one of the canonical targets of mTORC1 activation.

### Equivalents

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

### Sequences

All NCBI Gene and Accession Number Sequences are incorporated herein by reference in their entireties.

1. A method of inhibiting drusen formation in an ocular tissue, the method comprising administering to cells of the ocular tissue one or more inhibitors of Ribosomal protein S6 kinase beta-1 (S6K1).

2. The method of claim 1, wherein the ocular tissue comprises Bruch's membrane tissue, retinal pigment epithelium (RPE) tissue, macula tissue, or a combination thereof.

3. The method of claim 1, wherein the ocular tissue comprises photoreceptor cells, retinal pigment epithelial cells (RPEs), ganglion cells, or a combination thereof.

4. The method of claim 1, wherein the administration comprises topical administration, intravitreal administration, subconjunctival injection, intrachoroid injection, systemic injection, or any combination thereof.

5. The method of claim 1, wherein the at least one S6K1 inhibitor is a small molecule, peptide, protein, antibody, or inhibitory nucleic acid.

6. The method of claim 5, wherein the inhibitory nucleic acid is a dsRNA, siRNA, shRNA, miRNA, ami-RNA, antisense oligonucleotide (ASO), or aptamer.

7. The method of claim 5, wherein the inhibitory nucleic acid reduces or prevents expression of S6K1 protein.

8. The method of claim 5, wherein the inhibitory nucleic acid binds to a nucleic acid encoding a S6K1 protein.

9. The method of claim 1, wherein the protein is a dominant negative S6K1 protein.

10. The method of claim 1, wherein the small molecule is PF-4708671 rosmarinic acid methyl ester (RAME), A77 1726, or a salt, solvate, or analogue thereof.

11. The method of claim 10, wherein the small molecule is a selective inhibitor of S6K1.

12. The method of claim 1, wherein the S6K1 inhibitor does not bind to or inhibit expression or activity of mammalian target of rapamycin 1 (mTORC1).

13. The method of claim 1, wherein the administration reduces drusen formation by about 2-fold, 3-fold, 5-fold, 10-fold, 50-fold, 100-fold, or more than 100-fold in the ocular tissue relative to ocular tissue that has not been administered the one or more S6K1 inhibitor.

14. The method of claim 1, wherein the ocular tissue is in vivo, optionally wherein the ocular tissue is present in a subject's eye.

15. A method for treating age-related macular degeneration (AMD) in a subject, the method comprising administering to the subject one or more inhibitors of Ribosomal protein S6 kinase beta-1 (S6K1).

16. The method of claim 15, wherein the ocular tissue comprises Bruch's membrane tissue, retinal pigment epithelium (RPE) tissue, macula tissue, or a combination thereof.

17. The method of claim 15, wherein the ocular tissue comprises photoreceptor cells, retinal pigment epithelial cells (RPEs), ganglion cells, or a combination thereof.

18. The method of claim 15, wherein the administration comprises topical administration, intravitreal administration, subconjunctival injection, intrachoroid injection, systemic injection, or any combination thereof.

19. The method of claim 15, wherein the at least one S6K1 inhibitor is a small molecule, peptide, protein, antibody, or inhibitory nucleic acid.

20. The method of claim 19, wherein the inhibitory nucleic acid is a dsRNA, siRNA, shRNA, miRNA, ami-RNA, antisense oligonucleotide (ASO), or aptamer.

21. The method of claim 19, wherein the inhibitory nucleic acid reduces or prevents expression of S6K1 protein.

22. The method of claim 19, wherein the inhibitory nucleic acid binds to a nucleic acid encoding a S6K1 protein.

23. The method of claim 15, wherein the protein is a dominant negative S6K1 protein.

24. The method of claim 15, wherein the small molecule is PF-4708671, rosmarinic acid methyl ester (RAME), A77 1726, or a salt, solvate, or analogue thereof.

25. The method of claim 24, wherein the small molecule is a selective inhibitor of S6K1.

26. The method of claim 15, wherein the S6K1 inhibitor does not bind to or inhibit expression or activity of mammalian target of rapamycin 1 (mTORC1).

27. The method of claim 15, wherein the administration reduces drusen formation by about 2-fold, 3-fold, 5-fold, 10-fold, 50-fold, 100-fold, or more than 100-fold in the ocular tissue relative to ocular tissue that has not been administered the one or more S6K1 inhibitor.

28. The method of claim 15, wherein the ocular tissue is in vivo, optionally wherein the ocular tissue is present in a subject's eye.

29. The method of claim 15, the method further comprises administering to the subject an effective amount of di-docosahexaenoic acid (DHA).

30. The method of claim 29, wherein DHA is administered as dietary supplement.
